(19) United States
(12) Patent Application Publication
(10) Pub. No.: US 2023/0340064 A1
(43) Pub. Date: Oct. 26, 2023

(54) MAGE-A3 SPECIFIC T CELL RECEPTORS AND THEIR USE

(71) Applicant: MEDIGENE IMMUNOTHERAPIES GMBH, Planegg-Martinsried (DE)

(72) Inventors: Kathrin DAVARI, Poing (DE); Tristan HOLLAND, Muenchen (DE); Christian ELLINGER, Muenchen (DE)

(21) Appl. No.: 18/028,002

(22) PCT Filed: Sep. 24, 2021
(86) PCT No.: PCT/EP2021/076323

§ 371 Date: Mar. 23, 2023

(30) Foreign Application Priority Data
Sep. 24, 2020 (EP) 20198049.7

(51) Int. Cl. C07K 14/725; C12N 15/86; A61P 35/00

(52) U.S. Cl. CPC C07K 14/7051; C12N 15/86; A61P 35/00; C07K 2317/565; C07K 2317/35

(57) ABSTRACT

The present invention relates to an isolated T cell receptor (TCR) specific for a MAGE-A3-derived peptide and to a polypeptide comprising a functional portion of the TCR. Further implicated are a multivalent TCR complex, a nucleic acid sequence encoding a TCR, a cell expressing the TCR and a pharmaceutical composition comprising the TCR. The invention also refers to the TCR for use as a medicament, in particular to the TCR for use in the treatment of cancer.

### FIELD OF THE INVENTION

The present invention relates to isolated T cell receptors (TCR) specific for a MAGE-A3-derived peptide and to polypeptides comprising a functional portion of the TCR. Further implicated are a multivalent TCR complex, a nucleic acid encoding a TCR, a cell expressing the TCR and a pharmaceutical composition comprising the TCR. The invention also refers to the TCR for use as a medicament, in particular to the TCR for use in the treatment of cancer.

### BACKGROUND OF THE INVENTION

MAGE-A3, also called Melanoma-Associated Antigen 3, is a member of the MAGE-A gene family coding for a number of proteins having high homology with each other. The MAGE-A antigens belong to the family of cancer/testis antigens (CTA) and were the first human tumor-associated antigens identified at the molecular level (Science 1991. 254: 1643-1647/republished in J. Immunol. 2007; 178:2617-2621). The MAGE-A gene family includes 12 highly homologous genes located on chromosome Xq28. Their expression is consistently detected in cancers of different histological origin, such as non-small cell lung cancer, bladder cancer, esophageal, head and neck cancer and sarcoma, as well as myeloma, certain types of breast cancer and also in germinal cells (Front Med (Lausanne). 2017; 4: 18.). MAGE-A3 is a cytosolic/cytoplasmic protein and peptides derived from the MAGE-A3 protein are presented in an MEC-class I background, i.e. on human leukocyte antigen (HLA) molecules. More specifically, MAGE-A3 derived epitopes are presented on HLA-A1 molecules, indicating their suitability as promising targets for T cell-mediated cancer immunotherapy. The principle of cancer immunotherapy using adoptive cell transfer (ACT) enables a highly tumor specific cancer treatment utilizing a patient's own immune system. ACT uses ex vivo expanded autologous (patient-derived) T cells genetically engineered to express T cell receptors (TCR) with a specificity for a defined epitope derived from an intracellular protein like MAGE-A3.

In an open-label test of an affinity-enhanced TCR against HLA-A*01:01-restricted MAGE-A3 (EVDPIGHLY; SEQ ID NO: 1) severe side effects occurred. Studies revealed myocardial damage due to T cell infiltration of the heart tissue. Studies on the molecular level also revealed, that the TCR cross-reacted with a similar epitope of the striated muscle protein titin (Cameron, et al. Science Tranlational Medcine 5 (197): 1-11; 2013; Linette et al. Blood, 122(6), 863-871; 2013).

Thus, there is still a need for highly efficient TCRs which target only the tumor specific/restricted antigen MAGE-A3 and therefore bear an exceptional potential for cancer immunotherapy. Thus, there is a need for such specific TCRs targeting MAGE-A3 having no or only very low cross-reactivity to healthy tissue.

### OBJECTIVES AND SUMMARY OF THE INVENTION

One objective of the present invention is the provision of a T cell receptor (TCR) which is specific for MAGE-A3. In particular, the TCR may specifically recognize the MAGE-A3 epitope having the amino acid sequence SEQ ID NO: 1 or a fragment thereof. Preferably the TCR specifically recognizes the HLA-A1 bound form of the amino acid sequence of SEQ ID NO: 1, more preferably the TCR specifically recognizes the amino acid sequence of SEQ ID NO: 1, which is presented by the HLA-A*01:01 encoded molecule.

The fragment of the epitope may be a protein sequence that is specific for this antigen, i.e. does not occur in another protein or peptide of a mammal, especially of a human. The fragment may be shorter than the sequence of the antigen, such as at least 5%, at least 10%, at least 30%, at least 50%, at least 70%, at least 90% shorter than the antigen. The fragment may have a length of at least 7, at least 8, at least 9, at least 10, at least 11, at least 12, at least 13, at least 14, at least 15 or more amino acids.

In a specific embodiment, the TCR does not recognize the titin epitope of amino acid sequence SEQ ID NO: 54 or a fragment thereof. It is desired that there is no or substantially no cross-reactivity with the titin-derived epitope in order to avoid unwanted severe side effects.

It has been shown that a TCR described in the prior art directed against the epitope of SEQ ID NO: 1 led to severe side effects. It was also found that the adverse events arose due to the binding of the prior art TCR to the titin epitope of SEQ ID NO: 54, which is similar to the MAGE-A3 epitope of SEQ ID NO: 1. Therefore, it is highly important that a TCR, directed to MAGE-A3, in particular to the epitope of SEQ ID NO: 1, used for the treatment of humans is not activated by binding to the titin-derived epitopes, in particular to the titin epitope of SEQ ID NO: 54.

Preferably the TCR does not show cross-reactivity to other MAGE-A family members, specifically to MAGE-A6.

In a specific embodiment the isolated TCR comprises
a)—a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; or
b)—a TCR α chain comprising a CDR1 having the amino acid sequence of SEQ ID NO: 10, a CDR2 having the amino acid sequence of SEQ ID NO: 11 and a CDR3 having the amino acid sequence of SEQ ID NO: 12, and
   a TCR β chain comprising a CDR1 having the amino acid sequence of SEQ ID NO: 13, a CDR2 having the amino acid sequence of SEQ ID NO: 14 and a CDR3 having the amino acid sequence of SEQ ID NO: 15; or
c)—a TCR α chain comprising a CDR1 having the amino acid sequence of SEQ ID NO: 18, a CDR2 having the amino acid sequence of SEQ ID NO: 19 and a CDR3 having the amino acid sequence of SEQ ID NO: 20, and
   a TCR β chain comprising a CDR1 having the amino acid sequence of SEQ ID NO: 21, a CDR 2 having the amino acid sequence of SEQ ID NO: 22 and a CDR 3 having the amino acid sequence of SEQ ID NO: 23.

The TCR further may be defined by
a) a variable TCR α region having an amino acid sequence which is at least 80% identical to SEQ ID NO: 8 and a variable TCR β region having an amino acid sequence which is at least 80% identical to SEQ ID NO: 9; or
b) a variable TCR α region having an amino acid sequence which is at least 80% identical to SEQ ID NO: 16 and a variable TCR β region having an amino acid sequence which is at least 80% identical to SEQ ID NO: 17; or
c) a variable TCR α region having an amino acid sequence which is at least 80% identical to SEQ ID NO: 24 and a variable TCR β region having an amino acid sequence which is at least 80% identical to SEQ ID NO: 25.

The TCR according to the invention is isolated and/or purified and may be soluble or membrane bound.

In some embodiments, the amino acid sequence of the TCR may comprise one or more phenotypically silent substitutions. In addition, the TCR of the invention can be labelled with a detectable label. Additionally, or alternatively, the amino acid sequence may be modified to comprise a therapeutic agent or pharmacokinetic modifying moiety. The therapeutic agent may be selected from the group consisting of an immune effector molecule, a cytotoxic agent and a radionuclide. The immune effector molecule may for example be a cytokine. The pharmacokinetic modifying moiety may be at least one polyethylene glycol repeating unit, at least one glycol group, at least one sialyl group or a combination thereof.

The TCR, in particular a soluble form of the TCR, according to the invention can be modified by attaching additional functional moieties, e.g. for reducing immunogenicity, increasing hydrodynamic size (size in solution) solubility and/or stability (e.g. by enhanced protection to proteolytic degradation) and/or extending serum half-life. Other useful functional moieties and modifications include “suicide” or “safety switches” that can be used to shut off or turn on effector host cells carrying an inventive TCR in a patient's body or to shut off or turn on the transgenic TCR itself. TCRs with an altered glycosylation pattern are also envisaged herein.

It is also conceivable to add a drug or a therapeutic entity, such as a small molecule compound to the TCR, in particular to a soluble form of the inventive TCR.

The TCR, in particular a soluble form of the inventive TCR, can additionally be modified to introduce additional domains which aid in identification, tracking, purification and/or isolation of the respective molecule (tags).

In some embodiments, the TCR is of the single chain type, wherein the TCR α chain and the TCR β chain are linked by a linker sequence.

Another aspect of the invention refers to an isolated polypeptide comprising a functional portion of the TCR as described herein, wherein the functional portion comprises the
a) the amino acid sequences SEQ ID NOs: 2, 3, 4, 5, 6, and 7, or
b) the amino acid sequences SEQ ID NOs: 10, 11, 12, 13, 14, and 15, or
c) the amino acid sequences SEQ ID NOs:18, 19, 20, 21, 22, and 23.

In specific embodiments, the functional portion comprises the TCR α variable chain and/or the TCR β variable chain.

Further contemplated is a multivalent TCR complex comprising a least one TCR as described herein.

Thus, the invention refers to an isolated TCR as described herein, a polypeptide as described herein, a multivalent TCR complex as described herein, wherein IFN-γ secretion of a cell, e.g. T cell, which expresses the herein described TCR is induced by binding to the amino acid sequence of SEQ ID NO: 1, which is presented by the HLA-A*01:01 molecule.

Also contemplated is a nucleic acid encoding a TCR as described herein or encoding the isolated polypeptide as described herein.

Accordingly, another aspect refers to a vector comprising the nucleic acid as described herein, wherein the vector is preferably an expression vector, more preferably a retroviral vector or lentiviral vector.

Moreover, a further aspect defines a cell expressing the TCR as described herein.

Also envisaged is an antibody or antigen binding fragment thereof specifically binding to a portion of the TCR as described herein that mediates specificity for MAGE-A3, preferably wherein the portion of the TCR that mediates the MAGE-A3 specificity comprises
a) the CDR3 of the alpha chain of SEQ ID NO: 4 and/or the CDR3 of the beta chain of SEQ ID NO: 7 or;
b) the CDR3 of the alpha chain of SEQ ID NO: 12 and/or the CDR3 of the beta chain of SEQ ID NO: 15 or;
c) the CDR3 of the alpha chain of SEQ ID NO: 20 and/or the CDR3 of the beta chain of SEQ ID NO: 23.

Another aspect of the invention refers to a pharmaceutical composition comprising the TCR as described herein, the polypeptide as described herein, the multivalent TCR complex as described herein, the nucleic acid as described herein, the vector as described herein, the cell as described herein, or the antibody as described herein.

Typically, the pharmaceutical composition comprises at least one pharmaceutically acceptable carrier.

Another aspect refers to the TCR, the multivalent TCR complex, the nucleic acid, the vector, the cell, or the antibody as described herein for use as a medicament.

In particular, it is referred to the TCR, the multivalent TCR complex, the nucleic acid, the vector, the cell, or the antibody as described herein for use in the treatment of cancer, wherein the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and neck cancer, gastric cancer, endometrial cancer, cervical cancer, colorectal cancer, stomach adenocarcinoma, cholangiocarcinoma, breast cancer, bladder cancer, myeloid leukemia and acute lymphoblastic leukemia, carcinoma, sarcoma or osteosarcoma.

### FIGURE LEGENDS

FIG. 1 shows peptide selectivity of three different MAGE-A3-reactive TCRs. An in vitro priming approach was used to isolate MAGE-A3-reactive T cell clones and three individual representative clones expressing the same TCR are shown for each of the three different TCRs. T cell clones were co-cultured with DU-145 cells previously transduced with HLA-A1-GFP (DU-145_A1) either loaded with the relevant (EVD) or an irrelevant peptide (ESD) or electroporated with ivt-RNA encoding full length MAGE-A3. To analyze cytokine release, co-culture supernatants were harvested after 24 h and IFN-γ concentrations were analyzed by standard sandwich ELISA (BD human IFN-γ ELISA set).

FIG. 2 shows the MAGE-A3_{EVD}-MHC-multimer binding of T cells transduced with different MAGE-A3-reactive TCRs and untransduced T cells (UTD). CD8 T cells were isolated from PBMCs of a healthy donor and transduced with three different MAGE-A3-reactive TCRs. Transduced CD8⁺ T cells were enriched by FACS using the constant beta 1 region as a marker for transduction. After expansion of these cells, they were stained either with a MAGE-A3_{EVD}-MHC-multimer (right panel) or antibodies against CD8 and Cß1 (left panel) and analyzed by flow cytometry. Populations were gated on single live cells.

FIG. 3 shows that MAGE-A3-TCR-transgenic T cells selectively recognize MAGE-A3_{EVD} peptide endogenously processed and presented on HLA-A1. DU-145_A1 cells were further engineered to express mCherry alone (negative control), MAGE-A3-mCherry or MAGE-A6-mCherry. Untransduced (UTD) and transgenic T cells of three donors were co-cultured with transduced cells either unloaded or loaded with the relevant peptide (EVD). Recognition of target cells was analyzed by measuring the IFN-γ concentration in co-culture supernatants by a standard ELISA.

FIG. 4 shows that MAGE-A3-TCR-transgenic T cells recognize endogenous MAGE-A3-positive tumor cell lines. Untransduced (UTD) and transgenic T cells of three donors were co-cultured with endogenously MAGE-A3-positive or -negative tumor cell lines. Tumor cell lines were either endogenously HLA-A1-positive or engineered for HLA-A1 expression (TD). All tumor cell lines were tested either unloaded or loaded with the relevant peptide (EVD). Recognition of target cells was analyzed by measuring the IFN-γ concentration in co-culture supernatants by a standard ELISA.

### DETAILED DESCRIPTION OF THE INVENTION

Before the invention is described in detail with respect to some of its preferred embodiments, the following general definitions are provided.

The present invention as illustratively described in the following may be suitably practiced in the absence of any element or elements, limitation or limitations, not specifically disclosed herein.

The present invention will be described with respect to particular embodiments and with reference to certain figures, but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy 30 that the person skilled in the art will understand to still ensure the technical effect of the feature in question. The term typically indicates deviation from the indicated numerical value of ±10%, and preferably of ±5%.

Technical terms are used by their common sense or meaning to the person skilled in the art. If a specific meaning is conveyed to certain terms, definitions of terms will be given in the following in the context of which the terms are used.

TCR Background

A TCR is composed of two different and separate protein chains, namely the TCR alpha (a) and the TCR beta (β) chain. The TCR α chain comprises variable (V), joining (J) and constant (C) regions. The TCR β chain comprises variable (V), diversity (D), joining (J) and constant (C) regions. The rearranged V(D)J regions of both the TCR α and the TCR β chain contain hypervariable regions (CDR, complementarity determining regions), among which the CDR3 region determines the specific epitope recognition. At the C-terminal region both TCR α chain and TCR β chain contain a hydrophobic transmembrane domain and end in a short cytoplasmic tail.

Typically, the TCR is a heterodimer of one α chain and one β chain. This heterodimer can bind to MHC molecules presenting a peptide.

The term “variable TCR α region” or “TCR α variable chain” or “variable domain” in the context of the invention refers to the variable region of a TCR α chain. The term “variable TCR β region” or “TCR β variable chain” in the context of the invention refers to the variable region of a TCR β chain.

The TCR loci and genes are named using the International Immunogenetics (IMGT) TCR nomenclature (IMGT Database, www.IMGT.org; Giudicelli, V., et al. IMGT/LIGM-DB, the IMGT® comprehensive database of immunoglobulin and T cell receptor nucleotide sequences, Nucl. Acids Res., 34, D781-D784 (2006). PMID: 16381979; T cell Receptor Factsbook, LeFranc and LeFranc, Academic Press ISBN 0-12-441352-8).

Target

The target for the herein described TCR is MAGE-A3 (NCBI Reference Sequence: NP_005353.1-derived peptide EVD (SEQ ID NO: 1)

Thus, the TCR may specifically recognize the MAGE-A3 epitope having the amino acid sequence SEQ ID NO: 1 or a fragment thereof. Preferably, the TCR specifically recognizes the HLA-A1 bound form of the amino acid sequence of SEQ ID NO: 1, more preferably wherein the TCR specifically recognizes the amino acid sequence of SEQ ID NO: 1, which is presented by the HLA-A*01:01 encoded molecule.

The fragment may be a sequence of the antigen that is specific for this antigen, i.e. does not occur in another protein or peptide of a mammal, especially of a human. The fragment may be shorter than the sequence of the antigen, such as at least 5%, at least 10%, at least 30%, at least 50%, at least 70%, at least 90% shorter than the antigen. The fragment may have a length of at least 7, at least 8, at least 9, at least 10, at least 11, at least 12, at least 13, at least 14, at least 15 or more amino acids.

TCR Specific Sequence

Some embodiments relate to an isolated TCR comprising

a)—a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; or

b)—a TCR α chain comprising a CDR1 having the amino acid sequence of SEQ ID NO: 10, a CDR2 having the amino acid sequence of SEQ ID NO: 11 and a CDR3 having the amino acid sequence of SEQ ID NO: 12, and
a TCR β chain comprising a CDR1 having the amino acid sequence of SEQ ID NO: 13, a CDR2 having the amino acid sequence of SEQ ID NO: 14 and a CDR3 having the amino acid sequence of SEQ ID NO: 15; or
   c)—a TCR α chain comprising a CDR1 having the amino acid sequence of SEQ ID NO: 18, a CDR2 having the amino acid sequence of SEQ ID NO: 19 and a CDR3 having the amino acid sequence of SEQ ID NO: 20, and
a TCR β chain comprising a CDR1 having the amino acid sequence of SEQ ID NO: 21, a CDR 2 having the amino acid sequence of SEQ ID NO: 22 and a CDR 3 having the amino acid sequence of SEQ ID NO: 23.

In some embodiments, the TCR comprises the TCR comprises
a) a variable TCR α region having an amino acid sequence which is at least 80% identical to SEQ ID NO: 8 and a variable TCR β region having an amino acid sequence which is at least 80% identical to SEQ ID NO: 9; or
b) a variable TCR α region having an amino acid sequence which is at least 80% identical to SEQ ID NO: 16 and a variable TCR β region having an amino acid sequence which is at least 80% identical to SEQ ID NO: 17; or
c) a variable TCR α region having an amino acid sequence which is at least 80% identical to SEQ ID NO: 24 and a variable TCR β region having an amino acid sequence which is at least 80% identical to SEQ ID NO: 25.

“At least 80% identical”, in particular “having an amino acid sequence which is at least 80% identical” as used herein includes that the amino acid sequence is at least 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence set out.

The determination of percent identity between multiple sequences is preferably accomplished using the AlignX application of the Vector NTI Advance™ 10 program (Invitrogen Corporation, Carlsbad CA, USA). This program uses a modified Clustal W algorithm (Thompson et al., 1994. Nucl Acids Res. 22: pp. 4673-4680; Invitrogen Corporation; Vector NTI Advance™ 10 DNA and protein sequence analysis software. User's Manual, 2004, pp. 389-662). The determination of percent identity is performed with the standard parameters of the AlignX application.

A preferred embodiment relates to a TCR comprising
a) a variable TCR α region having the amino acid sequence of SEQ ID NO: 8 and a variable TCR β region having the amino acid sequence of SEQ ID NO: 9; or
b) a variable TCR α region having the amino acid sequence of SEQ ID NO: 16 and a variable TCR β region having the amino acid sequence of SEQ ID NO: 17; or
c) a variable TCR α region having the amino acid sequence of SEQ ID NO: 24 and a variable TCR β region having the amino acid sequence of SEQ ID NO: 25.

As can be seen from the examples the TCRs according to the invention are specific for MAGE-A3 and exhibit only very low cross-reactivity to other epitopes or antigens. In particular, the examples show that there is no substantial cross-reactivity to the similar titin epitope SEQ ID NO: 54. Moreover, there is no substantial cross-reactivity to other antigens of the MAGE-A family, such as MAGE-A6, as can be seen from the examples.

In specific embodiments the TCRs as described herein comprise a constant TCR α region having an amino acid sequence which is at least 80% identical to SEQ ID NO: 50 and a constant TCR region having an amino acid sequence which is at least 80% identical to SEQ ID NO: 51.

Hence more specifically, in some embodiments the TCR may comprise
a) a variable TCR α region having an amino acid sequence which is at least 80% identical to SEQ ID NO: 8, a variable TCR β region having an amino acid sequence which is at least 80% identical to SEQ ID NO: 9, a constant TCR α region having an amino acid sequence which is at least 80% identical to SEQ ID NO: 50 and a constant TCR β region having an amino acid sequence which is at least 80% identical to SEQ ID NO: 51; or
b) a variable TCR α region having an amino acid sequence which is at least 80% identical to SEQ ID NO: 16, a variable TCR β region having an amino acid sequence which is at least 80% identical to SEQ ID NO: 17 a constant TCR α region having an amino acid sequence which is at least 80% identical to SEQ ID NO: 50 and a constant TCR β region having an amino acid sequence which is at least 80% identical to SEQ ID NO: 51; or
c) a variable TCR α region having an amino acid sequence which is at least 80% identical to SEQ ID NO: 24 and a variable TCR β region having an amino acid sequence which is at least 80% identical to SEQ ID NO: 25, a constant TCR α region having an amino acid sequence which is at least 80% identical to SEQ ID NO: 50 and a constant TCR β region having an amino acid sequence which is at least 80% identical to SEQ ID NO: 51.

In even more specific embodiments, the TCR may comprise:
the TCR comprises
a) a variable TCR α region having the amino acid sequence of SEQ ID NO: 8, a variable TCR β region having the amino acid sequence of SEQ ID NO: 9, a constant TCR α region having the amino acid sequence of SEQ ID NO: 50 and a constant TCR β region having the amino acid sequence of SEQ ID NO: 51; or
b) a variable TCR α region having the amino acid sequence of SEQ ID NO: 16, a variable TCR region having the amino acid sequence of SEQ ID NO: 17, a constant TCR α region having the amino acid sequence of SEQ ID NO: 50 and a constant TCR β region having the amino acid sequence of SEQ ID NO: 51; or
c) a variable TCR α region having the amino acid sequence of SEQ ID NO: 24, a variable TCR β region having the amino acid sequence of SEQ ID NO: 25, a constant TCR α region having the amino acid sequence of SEQ ID NO: 50 and a constant TCR β region having the amino acid sequence of SEQ ID NO: 51.

The TCR according to the invention is isolated or purified. “Isolated” in the context of the invention means that the TCR is not present in the context in which it originally occurred in nature. “Purified” in the context of the invention means e.g. that the TCR is free or substantially free of other proteins and non-protein parts of the cell it originates from.

In some embodiments, the amino acid sequence of the TCR may comprise one or more phenotypically silent substitutions.

“Phenotypically silent substitutions” are also named “conservative amino acid substitutions”. The concept of “conservative amino acid substitutions” is understood by the skilled artisan, and preferably means that codons encoding positively-charged residues (H, K, and R) are substituted with codons encoding positively-charged residues, codons encoding negatively-charged residues (D and E) are substituted with codons encoding negatively-charged residues, codons encoding neutral polar residues (C, G, N, Q, S, T, and Y) are substituted with codons encoding neutral polar residues, and codons encoding neutral non-polar residues (A, F, I, L, M, P, V, and W) are substituted with codons encoding neutral non-polar residues. These variations can spontaneously occur, be introduced by random mutagenesis, or can be introduced by directed mutagenesis. Those changes can be made without destroying the essential characteristics of these polypeptides. The ordinarily skilled artisan can readily and routinely screen variant amino acids and/or the nucleic acids encoding them to determine if these variations substantially reduce or destroy the ligand binding capacity by methods known in the art.

The skilled person understands that also the nucleic acid encoding the TCR may be modified. Useful modifications in the overall nucleic acid sequence include codon optimization of the sequence. Alterations may be made which lead to conservative substitutions within the expressed amino acid sequence. These variations can be made in complementarity determining and non-complementarity determining regions of the amino acid sequence of the TCR chain that do not affect function. Usually, additions and deletions should not be performed in the CDR3 region.

According to some embodiments of the invention the amino acid sequence of the TCR is modified to comprise a detectable label, a therapeutic agent or pharmacokinetic modifying moiety.

Non-limiting examples for detectable labels are radiolabels, fluorescent labels, nucleic acid probes, enzymes and contrast reagents. Therapeutic agents which may be associated with the TCRs include radioactive compounds, immune-modulators, enzymes or chemotherapeutic agents. The therapeutic agents could be enclosed by a liposome linked to TCR so that the compound can be released slowly at the target site. This will avoid damage during the transport in the body and ensure that the therapeutic agent, e.g. toxin, has maximum effect after binding of the TCR to the relevant antigen presenting cells. Other examples for therapeutic agents are: peptide cytotoxins, i.e. proteins or peptides with the ability to kill mammalian cells, such as ricin, diphtheria toxin, pseudomonas bacterial exotoxin A, DNase and RNase. Small molecule cytotoxic agents, i.e. compounds with the ability to kill mammalian cells having a molecular weight of less than 700 Daltons. Such compounds could contain toxic metals capable of having a cytotoxic effect. Furthermore, it is to be understood that these small molecule cytotoxic agents also include pro-drugs, i.e. compounds that decay or are converted under physiological conditions to release cytotoxic agents. Such agents may for example include docetaxel, gemcitabine, cisplatin, maytansine derivatives, rachelmycin, calicheamicin, etoposide, ifosfamide, irinotecan, porfimer sodium photofrin II, temozolomide, topotecan, trimetrexate glucoronate, mitoxantrone, auristatin E, vincristine and doxorubicin; radionuclides, such as, iodine 131, rhenium 186, indium 111, yttrium 90. bismuth 210 and 213, actinium 225 and astatine 213. The association of the radionuclides with the TCRs or derivatives thereof may for example be carried out by chelating agents; immune-stimulators, also known as immunostimulants, i.e. immune effector molecules which stimulate immune response. Exemplary immune-stimulators are cytokines such as IL-2 and IFN-γ, antibodies or fragments thereof, including anti-T cell or NK cell determinant antibodies (e.g anti-CD3, anti-CD28 or anti-CD16); alternative protein scaffolds with antibody like binding characteristics; Superantigens, i.e. antigens that cause non-specific activation of T-cells resulting in polyclonal T cell activation and massive cytokine release, and mutants thereof; chemokines such as IL-8, platelet factor 4, melanoma growth stimulatory protein, etc. complement activators; xenogeneic protein domains, allogeneic protein domains, viral/bacterial protein domains, viral/bacterial peptides.

The antigen receptor molecules (T cell receptor molecules) on human T lymphocytes are non-covalently associated with the CD3 (T3) molecular complex on the cell surface. Perturbation of this complex with anti-CD3 monoclonal antibodies induces T cell activation. Thus, some embodiments refer to a TCR as described herein associated (usually by fusion to an N- or C-terminus of the alpha or beta chain) with an anti-CD3 antibody, or a functional fragment or variant of said anti-CD3 antibody. Antibody fragments and variants/analogues which are suitable for use in the compositions and methods described herein include minibodies, Fab fragments, F(ab<′>)2fragments, dsFv and scFv fragments, Nanobodies™ (Ablynx (Belgium), molecules comprising synthetic single immunoglobulin variable heavy chain domain derived from a camelid (e.g. camel or llama) antibody) and Domain Antibodies (comprising an affinity matured single immunoglobulin variable heavy chain domain or immunoglobulin variable light chain domain (Domantis (Belgium)) or alternative protein scaffolds that exhibit antibody-like binding characteristics such as Affibodies (comprising engineered protein A scaffold Affibody (Sweden)) or Anticalins (comprising engineered anticalins Pieris (German)).

The therapeutic agent may preferably be selected from the group consisting of an immune effector molecule, a cytotoxic agent and a radionuclide. Preferably, the immune effector molecule is a cytokine.

The pharmacokinetic modifying moiety may be for example at least one polyethylene glycol repeating unit, at least one glycol group, at least one sialyl group or a combination thereof. The association of at least one polyethylene glycol repeating unit, at least one glycol group, at least one sialyl group may be caused in a number of ways known to those skilled in the art. In a preferred embodiment the units are covalently linked to the TCR. The TCRs according to the invention can be modified by one or several pharmacokinetic modifying moieties. In particular, the soluble form of the TCR is modified by one or several pharmacokinetic modifying moieties. The pharmacokinetic modifying moiety may achieve beneficial changes to the pharmacokinetic profile of the therapeutic, for example improved plasma half-life, reduced or enhanced immunogenicity, and improved solubility.

The TCR according to the invention may be soluble or membrane bound. The term “soluble” refers to a TCR being in soluble form (i.e. having no transmembrane or cytoplasmic domains), for example for use as a targeting agent for delivering therapeutic agents to the antigen presenting cell. For stability, soluble αβ heterodimeric TCRs preferably have an introduced disulfide bond between residues of the respective constant domains, as described, for example, in WO 03/020763. One or both of the constant domains present in an αβ heterodimer of the invention may be truncated at the C terminus or C termini, for example by up to 15, or up to 10 or up to 8 or fewer amino acids. For use in adoptive therapy, an αβ heterodimeric TCR may, for example, be transfected as full-length chains having both cytoplasmic and transmembrane domains. TCRs may contain a disulfide bond corresponding to that found in nature between the respective alpha and beta constant domains, additionally or alternatively a non-native disulfide bond may be present.

The TCR, in particular a soluble form of the TCR according to the invention, can thus be modified by attaching additional functional moieties, e.g. for reducing immunogenicity, increasing hydrodynamic size (size in solution) solubility and/or stability (e.g. by enhanced protection to proteolytic degradation) and/or extending serum half-life.

Other useful functional moieties and modifications include “suicide” or “safety switches” that can be used to shut off effector host cells carrying an inventive TCR in a patient's body. An example is the inducible Caspase 9 (iCasp9) “safety switch” described by Gargett and Brown Front Pharmacol. 2014; 5: 235. Briefly, effector host cells are modified by well-known methods to express a Caspase 9 domain whose dimerization depends on a small molecule dimerizer drug such as AP1903/CIP, and results in rapid induction of apoptosis in the modified effector cells. The system is for instance described in EP2173869 (A2). Examples for other “suicide” “safety switches” are known in the art, e.g. Herpes Simplex Virus thymidine kinase (HSV-TK), expression of CD20 and subsequent depletion using anti-CD20 antibody or myc tags (Kieback et al, Proc Natl Acad Sci USA. 2008 Jan. 15; 105(2):623-8).

TCRs with an altered glycosylation pattern are also envisaged herein. As is known in the art, glycosylation patterns can depend on the amino acid sequence (e.g., the presence or absence of particular glycosylation amino acid residues, discussed below) and/or the host cell or organism in which the protein is produced. Glycosylation of polypeptides is typically either N-linked or 0-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of or substitution by, one or more serine or threonine residues to the starting sequence. Another means of glycosylation of TCRs is by chemical or enzymatic coupling of glycosides to the protein. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. Similarly, deglycosylation (i.e., removal of carbohydrate moieties present on the binding molecule) may be accomplished chemically, e.g. by exposing the TCRs to trifluoromethanesulfonic acid, or enzymatically by employing endo- and exo-glycosidases.

It is also conceivable to add a drug such as a small molecule compound to the TCR, in particular a soluble form of the inventive TCR. Linkage can be achieved via covalent bonds, or non-covalent interactions such as through electrostatic forces. Various linkers, known in the art, can be employed in order to form the drug conjugates.

The TCR, in particular a soluble form of the inventive TCR can additionally be modified to introduce additional domains which aid in identification, tracking, purification and/or isolation of the respective molecule (tags). Thus, in some embodiments, the TCR α chain or the TCR β chain may be modified to comprise an epitope tag.

Epitope tags are useful examples of tags that can be incorporated into the TCR of the invention. Epitope tags are short stretches of amino acids that allow for binding of a specific antibody and therefore enable identification and tracking of the binding and movement of soluble TCRs or host cells within the patient's body or cultivated (host) cells. Detection of the epitope tag, and hence, the tagged TCR, can be achieved using a number of different techniques.

Tags can further be employed for stimulation and expansion of host cells carrying an inventive TCR by cultivating the cells in the presence of binding molecules (antibodies) specific for said tag.

In general, the TCR can be modified in some instances with various mutations that modify the affinity and the off-rate of the TCR with the target antigen. In particular, the mutations may increase the affinity and/or reduce the off-rate. Thus, the TCR may be mutated in at least one CDR and the variable domain framework region thereof.

However, in a preferred embodiment the CDR regions of the TCR are not modified or in vitro affinity maturated such as for the TCR receptors in the examples. This means that the CDR regions have naturally occurring sequences. This can be advantageous, since in vitro affinity maturation may lead to immunogenicity to the TCR molecule. This may lead to the production of anti-drug antibodies decreasing or inactivating the therapeutic effect and the treatment and/or induce adverse effects.

The mutation may be one or more substitution(s), deletion(s) or insertions(s). These mutations may be introduced by any suitable method known in the art, such as polymerase chain reaction, restriction enzyme-based cloning, ligation independent cloning procedures, which are described for Example in Sambrook, Molecular Cloning—4^{th} Edition (2012) Cold Spring Harbor Laboratory Press.

Theoretically, unpredictable TCR specificity with the risk for cross-reactivity can occur due to mispairing between endogenous and exogenous TCR chains. To avoid mispairing of TCR sequences, the recombinant TCR sequence may be modified to contain minimal murinized Ca and CP regions, a technology that has been shown to efficiently enhance correct pairing of several different transduced TCR chains. Murinization of TCRs (i.e. exchanging the human constant regions in the alpha and beta chain by their murine counterparts) is a technique that is commonly applied in order to improve cell surface expression of TCRs in host cells. Without wishing to be bound by specific theory, it is thought that murinized TCRs associate more effectively with CD3 co-receptors; and/or that preferentially pair with each other and are less prone to form mixed TCRs on human T cells genetically modified ex vivo to express the TCRs of desired antigenic specificity, but still retaining and expressing their “original” TCRs.

Nine amino acids responsible for the improved expression of murinized TCRs have been identified (Sommermeyer and Uckert, J Immunol. 2010 Jun. 1; 184(11):6223-31) and it is envisaged to substitute one or all of the amino acid residues in the TCRs alpha and/or beta chain constant region for their murine counterpart residues. This technique is also referred to as “minimal murinization”, and offers the advantage of enhancing cell surface expression while, at the same time, reducing the number of “foreign” amino acid residues in the amino acid sequence and, thereby, the risk of immunogenicity.

Some embodiments refer to an isolated TCR as described herein, wherein the TCR is of the single chain type, wherein the TCR α chain and the TCR β chain are linked by a linker sequence.

A suitable single chain TCR form comprises a first segment constituted by an amino acid sequence corresponding to a variable TCR α region, a second segment constituted by an amino acid sequence corresponding to a variable TCR β region fused to the N terminus of an amino acid sequence corresponding to a TCR β chain constant region extracellular sequence, and a linker sequence linking the C terminus of the first segment to the N terminus of the second segment. Alternatively, the first segment may be constituted by an amino acid sequence corresponding to a TCR β chain variable region, the second segment may be constituted by an amino acid sequence corresponding to a TCR α chain variable region sequence fused to the N terminus of an amino acid sequence corresponding to a TCR α chain constant region extracellular sequence. The above single chain TCRs may further comprise a disulfide bond between the first and second chains, and wherein the length of the linker sequence and the position of the disulfide bond being such that the variable domain sequences of the first and second segments are mutually orientated substantially as in native T cell receptors. More specifically the first segment may be constituted by an amino acid sequence corresponding to a TCR α chain variable region sequence fused to the N terminus of an amino acid sequence corresponding to a TCR α chain constant region extracellular sequence, the second segment may be constituted by an amino acid sequence corresponding to a TCR β chain variable region fused to the N terminus of an amino acid sequence corresponding to TCR β chain constant region extracellular sequence, and a disulfide bond may be provided between the first and second chains. The linker sequence may be any sequence which does not impair the function of the TCR.

In the context of the present invention, a “functional” TCR α and/or β chain fusion protein shall mean a TCR or TCR variant, for example modified by addition, deletion or substitution of amino acids, that maintains at least substantial biological activity. In the case of the α and/or β chain of a TCR, this shall mean that both chains remain able to form a T-cell receptor (either with a non-modified α and/or β chain or with another inventive fusion protein α and/or β chain) which exerts its biological function, in particular binding to the specific peptide-MHC complex of said TCR, and/or functional signal transduction upon specific peptide:MHC interaction.

In specific embodiments the TCR may be modified, to be a functional T-cell receptor (TCR) α and/or β chain fusion protein, wherein said epitope-tag has a length of between 6 to 15 amino acids, preferably 9 to 11 amino acids. In another embodiment the TCR may be modified to be a functional T-cell receptor (TCR) α and/or β chain fusion protein wherein said T-cell receptor (TCR) α and/or β chain fusion protein comprises two or more epitope-tags, either spaced apart or directly in tandem. Embodiments of the fusion protein can contain 2, 3, 4, 5 or even more epitope-tags, as long as the fusion protein maintains its biological activity/activities (“functional”).

Preferred is a functional T-cell receptor (TCR) α and/or β chain fusion protein according to the present invention, wherein said epitope-tag is selected from, but not limited to, CD20 or Her2/neu tags, or other conventional tags such as a myc-tag, FLAG-tag, T7-tag, HA (hemagglutinin)-tag, His-tag, S-tag, GST-tag, or GFP-tag. myc, T7, GST, GFP tags are epitopes derived from existing molecules. In contrast, FLAG is a synthetic epitope tag designed for high antigenicity (see, e.g., U.S. Pat. Nos. 4,703,004 and 4,851,341). The myc tag can preferably be used because high quality reagents are available to be used for its detection. Epitope tags can of course have one or more additional functions, beyond recognition by an antibody. The sequences of these tags are described in the literature and well known to the person of skill in art.

TCR Variants

Another aspect of the invention refers to a polypeptide comprising a functional portion of the TCR of as described herein, wherein the functional portion comprises
a) the amino acid sequences SEQ ID NOs: 2, 3, 4, 5, 6, and 7, or
b) the amino acid sequences SEQ ID NOs: 10, 11, 12, 13, 14, and 15, or
c) the amino acid sequences SEQ ID NOs: 18, 19, 20, 21, 22, and 23.

The functional portion may mediate the binding of the TCR to the antigen, in particular to the antigen-MHC complex.

In one embodiment, the functional portion comprises the TCR α variable chain and/or the TCR β variable chain as described herein.

The TCR variant molecule may have the binding properties of the TCR receptor but may be combined with signaling domains of effectors cells (other than T cells), in particular with signaling domains of NK cells. Therefore, some embodiments refer to a protein comprising a functional portion of the TCR as described herein in combination with the signaling domains of an effector cell, such as a NK cell.

Another aspect of the invention refers to a multivalent TCR complex comprising at least one TCR, preferably at least two TCRs as described herein. In one embodiment of this aspect, at least two TCR molecules are linked via linker moieties to form multivalent complexes. Preferably, the complexes are water soluble, so the linker moiety should be selected accordingly. It is preferable that the linker moiety is capable of attaching to defined positions on the TCR molecules, so that the structural diversity of the complexes formed is minimized. One embodiment of the present aspect is provided by a TCR complex of the invention wherein the polymer chain or peptidic linker sequence extends between amino acid residues of each TCR which are not located in a variable region sequence of the TCR. Since the complexes of the invention may be for use in medicine, the linker moieties should be chosen with due regard to their pharmaceutical suitability, for example their immunogenicity. Examples of linker moieties which fulfil the above desirable criteria are known in the art, for example the art of linking antibody fragments.

Examples for linkers are hydrophilic polymers and peptide linkers. An example for hydrophilic polymers are polyalkylene glycols. The most commonly used of this class are based on polyethylene glycol or PEG. However, others are based on other suitable, optionally substituted, polyalkylene glycols which include polypropylene glycol, and copolymers of ethylene glycol and propylene glycol. Peptide linkers are comprised of chains of amino acids, and function to produce simple linkers or multimerization domains onto which TCR molecules can be attached.

One embodiment refers to a multivalent TCR complex, wherein at least one of said TCRs is associated with a therapeutic agent.

Cytokine and Chemokine Release

Some embodiments refer to the isolated TCR as described herein, polypeptide as described herein, multivalent TCR complex as described herein, wherein IFN-γ secretion is induced by binding of the inventive TCR expressed on an effector cell to the HLA-A*01 bound form of the amino acid sequence selected from the group consisting of SEQ ID NO: 1.

The IFN-γ secretion induced by binding of the inventive TCR expressed on an effector cell to the HLA-A*01 bound form of the amino acid sequence of SEQ ID NO:1 may be more than 500 pg/ml, more preferably more than 1000 pg/ml, most preferably more than 2000 pg/ml. The IFN-γ secretion may be at least 5 times higher when binding to the HLA-A*01 bound form of the amino acid sequence of SEQ ID NO: 1 compared to binding to the HLA-A*01 bound form of an irrelevant peptide (e.g. SEQ ID No: 54).

According. “wherein the TCR does not recognize the amino acid sequence” means that the the IFN-γ secretion of the inventive TCR when contacted the non-target amino acid is less than 300 pg/ml, less than 200 pg/ml, less than 100 pg/ml. In particular the IFN-γ secretion of the inventive TCR when contacted with the titin epitope of SEQ ID NO: 54 is less than than 300 pg/ml, preferred less than 200 pg/ml, more preferred less than 100 pg/ml.

The “effector cell” may be a peripheral blood lymphocyte (PBL) or a peripheral blood mononuclear cell (PBMC). Typically, the effector cell is an immune effector cell, especially a T cell. Other suitable cell types include gamma-delta T cells and NK-like T cells.

The invention relates also to methods for identifying a TCR or a fragment thereof that binds to the target amino acid sequence SEQ ID NO: 1 or the HLA-A*01, preferably or the HLA-A*01:01 bound form thereof, wherein the method comprises contacting the candidate TCR or antigen binding fragment thereof with the amino acid sequences of SEQ ID NO: 1 or the HLA-A*01, preferably or the HLA-A*01:01 bound form thereof and determining whether the candidate TCR or antigen binding fragment thereof binds to the target and/or mediates an immune response. Whether the candidate TCR or antigen binding fragment thereof mediates an immune response can be determined for example by the measurement of cytokine secretion, such as IFN-γ secretion. Cytokine secretion may be measured by an in vitro assay in which DU-145 cells, transduced with HLA-A1 (or other APCs transduced with HLA-A1) transfected with ivtRNA coding for the amino acid sequence SEQ ID NO: 1 are incubated with CD8+ enriched PBMC expressing the TCR or a molecule comprising a fragment of the TCR to be investigated.

The TCR of the invention is particularly useful since it shows high tumor cell recognition capacity. Further the TCR as described herein exhibits high tumor cell killing capacity. In addition, the TCR of the invention has a high functional avidity.

Moreover, the TCR shows favourable cytokine release pattern, which is advantageous for effective tumor regression.

Nucleic Acids, Vectors

Another aspect of the invention refers to a nucleic acid encoding a TCR as described herein or encoding the polynucleotide encoding a TCR as described herein.

| Peptide | nucleotide | |
|---|---|---|
| seq. | seq. | |
| SEQ ID NO | SEQ ID NO | description |
| 2 | 26 | TCR1 α chain CDR1 |
| 3 | 27 | TCR1 α chain CDR2 |
| 4 | 28 | TCR1 α chain CDR3 |
| 5 | 29 | TCR1 β chain CDR1 |
| 6 | 30 | TCR1 β chain CDR2 |
| 7 | 31 | TCR1 β chain CDR3 |
| 8 | 32 | TCR1 α chain variable region |
| 9 | 33 | TCR1 β chain variable region |
| 10 | 34 | TCR2 α chain CDR1 |
| 11 | 35 | TCR2 α chain CDR2 |
| 12 | 36 | TCR2 α chain CDR3 |
| 13 | 37 | TCR2 β chain CDR1 |
| 14 | 38 | TCR2 β chain CDR2 |
| 15 | 39 | TCR2 β chain CDR3 |
| 16 | 40 | TCR2 α chain variable region |
| 17 | 41 | TCR2 β chain variable region |
| 18 | 42 | TCR3 α chain CDR1 |
| 19 | 43 | TCR3 α chain CDR2 |
| 20 | 44 | TCR3 α chain CDR3 |
| 21 | 45 | TCR3 β chain CDR1 |
| 22 | 46 | TCR3 β chain CDR2 |
| 23 | 47 | TCR3 β chain CDR3 |
| 24 | 48 | TCR3 α chain variable region |
| 25 | 49 | TCR3 β chain variable region |
| 50 | 52 | TCR α chain constant region |
| | | (sequences apply to TCR 1-3) |
| 51 | 53 | TCR β chain constant region |
| | | (sequences apply to TCR 1-3) |

“Nucleic acid molecule” and generally means a polymer of DNA or RNA, which can be single-stranded or double-stranded, synthesized or obtained (e.g., isolated and/or purified) from natural sources which can contain natural, non-natural or altered nucleotides, and which can contain a natural, non-natural or altered internucleotide linkage, such as a phosphoroamidate linkage or a phosphorothioate linkage instead of the phosphodiester found between the nucleotides of an unmodified oligonucleotide. Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids can be constructed based on chemical synthesis and/or enzymatic ligation reactions using procedures known in the art or commercially available (e.g. from Genscript, Thermo Fisher and similar companies). See, for example, Sambrook et al. for example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). The nucleic acid can comprise any nucleotide sequence which encodes any of the recombinant TCRs, polypeptides, or proteins, or functional portions or functional variants thereof.

The present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence encoding the TCR described herein. Such variant nucleotide sequence encodes a functional TCR that specifically recognizes MAGE-A3.

The disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand and are particularly suitable for detecting expression of any of the TCRs described herein. It is generally appreciated that conditions can be rendered more stringent by the addition of increasing amounts of formamide.

As already described elsewhere herein, the nucleic acid encoding the TCR may be modified. Useful modifications in the overall nucleic acid sequence may be codon optimization. Alterations may be made which lead to conservative substitutions within the expressed amino acid sequence. These variations can be made in complementarity determining and non-complementarity determining regions of the amino acid sequence of the TCR chain that do not affect function. Usually, additions and deletions should not be performed in the CDR3 region.

Another embodiment refers to a vector comprising the nucleic acid encoding the TCR as described herein.

The vector is preferably a plasmid, shuttle vector, phagemide, cosmid, expression vector, retroviral vector, adenoviral vector or particle and/or vector to be used in gene therapy.

A “vector” is any molecule or composition that has the ability to carry a nucleic acid sequence into a suitable host cell where synthesis of the encoded polypeptide can take place. Typically, and preferably, a vector is a nucleic acid that has been engineered, using recombinant DNA techniques that are known in the art, to incorporate a desired nucleic acid sequence (e.g. a nucleic acid of the invention). The vector may comprise DNA or RNA and/or comprise liposomes. The vector may be a plasmid, shuttle vector, phagemide, cosmid, expression vector, retroviral vector, lentiviral vector, adenoviral vector or particle and/or vector to be used in gene therapy. A vector may include nucleic acid sequences that permit it to replicate in a host cell, such as an origin of replication. A vector may also include one or more selectable marker genes and other genetic elements known to those of ordinary skill in the art. A vector preferably is an expression vector that includes a nucleic acid according to the present invention operably linked to sequences allowing for the expression of said nucleic acid.

Preferably, the vector is an expression vector. More preferably, the vector is a retroviral, more specifically a gamma-retroviral or lentiviral vector.

Cells, Cell Lines

Another aspect of the invention refers to a cell expressing the TCR as described herein.

In some embodiments, the cell is isolated or non-naturally occurring.

In specific embodiments, the cell may comprise the nucleic acid encoding the TCR as described herein or the vector comprising said nucleic acid.

In the cell the above described vector comprising a nucleic acid sequence coding for the above described TCR may be introduced or ivtRNA coding for said TCR may be introduced. The cell may be a peripheral blood lymphocyte such as a T cell. The method of cloning and exogenous expression of the TCR is for example described in Engels et al. (Relapse or eradication of cancer is predicted by peptide-major histocompatibility complex affinity. Cancer Cell, 23(4), 516-26. 2013). The transduction of primary human T cells with a lentiviral vector is, for example, described in Cribbs “simplified production and concentration of lentiviral vectors to achieve high transduction in primary human T cells” BMC Biotechnol. 2013; 13: 98.

The term “transfection” and “transduction” are interchangeable and refer to the process by which an exogenous nucleic acid sequence is introduced in a host cell, e.g. in an eukaryotic host cell. It is noted that introduction or transfer of nucleic acid sequences is not limited to the mentioned methods but can be achieved by any number of means including electroporation, microinjection, gene gun delivery, lipofection, superfection and the mentioned infection by retroviruses or other suitable viruses for transduction or transfection.

Some embodiments refer to a cell comprising:
a) an expression vector which comprises at least one nucleic acid as described herein, or
b) a first expression vector which comprises a nucleic acid encoding the alpha chain of the TCR as described herein, and a second expression vector which comprises a nucleic acid encoding the beta chain of a TCR as described herein.

In some embodiments, the cell is a peripheral blood lymphocyte (PBL) or a peripheral blood mononuclear cell (PBMC). The cell may be a natural killer cell or a T cell. Preferably, the cell is a T cell. The T cell may be a CD4+ or a CD8+ T cell. In some embodiments the cell is a stem cell like memory T cell.

Stem cell-like memory T cells (TSCM) are a less-differentiated subpopulation of CD8+ or CD4+ T cells, which are characterized by the capacity of self-renewal and to persist long-term. Once these cells encounter their antigen in vivo, they differentiate further into central memory T cells (TCM), effector memory T cells (TEM) and terminally differentiated effector memory T cells (TEMRA) with some TSCM remaining quiescent (Flynn et al., Clinical & Translational Immunology (2014). These remaining TSCM cells show the capacity to build a durable immunological memory in vivo and therefore are considered an important T cell subpopulation for adoptive T cell therapy (Lugli et al., Nature Protocols 8, 33-42 (2013) Gattinoni et al., Nat. Med. 2011 October; 17(10): 1290-1297). Immune-magnetic selection can be used in order to restrict the T cell pool to the stem cell memory T cell subtype see (Riddell et al. 2014, Cancer Journal 20(2): 141-44)

Antibodies Targeting TCR

Another aspect of the invention refers to an antibody or antigen binding fragment thereof specifically binding to a portion of the TCR as described herein, wherein the the portion of the TCR comprises
a) CDR3 of the alpha chain of SEQ ID NO: 4 and/or the CDR3 of the beta chain of SEQ ID NO: 7 or;
b) CDR3 of the alpha chain of SEQ ID NO: 12 and/or the CDR3 of the beta chain of SEQ ID NO: 15 or;
c) CDR3 of the alpha chain of SEQ ID NO: 20 and/or the CDR3 of the beta chain of SEQ ID NO: 23.

In particular, the invention refers to an antibody or antigen binding fragment thereof specifically binding to a portion of the TCR as described herein that mediates specificity for MAGE-A3. In one embodiment, the portion of the TCR that mediates the MAGE-A3 specificity comprises
a) (i) the CDR1 of the alpha chain of SEQ ID NO: 2, the CDR3 of the alpha chain of SEQ ID NO: 4; and/or
   (ii) the CDR1 of the beta chain of SEQ ID NO: 5 the CDR3 of the beta chain of SEQ ID NO: 7;
or;
b) (i) the CDR1 of the alpha chain of SEQ ID NO: 10, the CDR3 of the alpha chain of SEQ ID NO: 12; and/or
   (ii) the CDR1 of the beta chain of SEQ ID NO: 13, the CDR3 of the beta chain of SEQ ID NO: 15;
or;
c) (i) the CDR1 of the alpha chain of SEQ ID NO: 18, the CDR3 of the alpha chain of SEQ ID NO: 20; and/or
   (ii) the CDR1 of the beta chain of SEQ ID NO: 21, the CDR3 of the beta chain of SEQ ID NO: 23.

In some embodiments, the invention refers to an antibody or antigen binding fragment thereof specifically binding to a portion of the TCR as described herein that mediates specificity for MAGE-A3. In one embodiment, the portion of the TCR that mediates the MAGE-A3 specificity comprises
a) (i) the CDR1 of the alpha chain of SEQ ID NO: 2,
   the CDR2 of the alpha chain of SEQ ID NO: 3,
   the CDR3 of the alpha chain of SEQ ID NO: 4; and/or
   (ii) the CDR1 of the beta chain of SEQ ID NO: 5
   the CDR2 of the alpha chain of SEQ ID NO: 6,
   the CDR3 of the beta chain of SEQ ID NO: 7
   or;
b) (i) the CDR1 of the alpha chain of SEQ ID NO: 10
   the CDR2 of the alpha chain of SEQ ID NO: 11,
   the CDR3 of the alpha chain of SEQ ID NO: 12 and/or
   (ii) the CDR1 of the beta chain of SEQ ID NO: 13
   the CDR2 of the beta chain of SEQ ID NO: 14,
   the CDR3 of the beta chain of SEQ ID NO: 15
   or;
c) (i) the CDR1 of the alpha chain of SEQ ID NO: 18
   the CDR2 of the alpha chain of SEQ ID NO: 19,
   the CDR3 of the alpha chain of SEQ ID NO: 20 and/or
   (ii) the CDR1 of the beta chain of SEQ ID NO: 21
   the CDR2 of the beta chain of SEQ ID NO: 22
   the CDR3 of the beta chain of SEQ ID NO: 23.

The antibody antigen binding fragment may modulate the activity of the TCR. It may block or may not block the binding of the TCR with MAGE-A3. It could be used for modulating the therapeutic activity of the TCR or for diagnostic purposes.

Pharmaceutical Compositions, Medical Treatments and Kits

Another aspect of the invention refers to pharmaceutical composition comprising the TCR as described herein, the polypeptide comprising a functional portion of said TCR, the multivalent TCR complex as described herein, the nucleic acid encoding the TCR, the vector comprising said nucleic acid, the cell comprising said TCR, or the antibody specifically binding to a portion of the TCR as described herein.

Those active components of the present invention are preferably used in such a pharmaceutical composition, in doses mixed with an acceptable carrier or carrier material, that the disease can be treated or at least alleviated. Such a composition can (in addition to the active component and the carrier) include filling material, salts, buffer, stabilizers, solubilizers and other materials, which are known state of the art.

The term “pharmaceutically acceptable” defines a non-toxic material, which does not interfere with effectiveness of the biological activity of the active component. The choice of the carrier is dependent on the application.

The pharmaceutical composition may contain additional components which enhance the activity of the active component or which supplement the treatment. Such additional components and/or factors can be part of the pharmaceutical composition to achieve synergistic effects or to minimize adverse or unwanted effects.

Techniques for the formulation or preparation and application/medication of active components of the present invention are published in “Remington's Pharmaceutical Sciences”, Mack Publishing Co., Easton, PA, latest edition. An appropriate application is a parenteral application, for example intramuscular, subcutaneous, intramedular injections as well as intrathecal, direct intraventricular, intravenous, intranodal, intraperitoneal or intratumoral injections. The intravenous injection is the preferred treatment of a patient.

According to a preferred embodiment, the pharmaceutical composition is an infusion or an injection.

An injectable composition is a pharmaceutically acceptable fluid composition comprising at least one active ingredient, e.g. an expanded T cell population (for example autologous or allogenic to the patient to be treated) expressing a TCR. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the fusion proteins of this disclosure are conventional; appropriate formulations are well known to those of ordinary skill in the art.

Typically, the pharmaceutical composition comprises at least one pharmaceutically acceptable carrier.

Accordingly, another aspect of the invention refers to the TCR as described herein, the polypeptide comprising a functional portion of said TCR, the multivalent TCR complex according as described herein, the nucleic acid encoding said TCR, the vector comprising said nucleic acid, the cell comprising said TCR, or the antibody specifically binding to a portion of the TCR as described herein for use as a medicament.

Some embodiments refer to the TCR as described herein, the polypeptide comprising a functional portion of said TCR, the multivalent TCR complex according as described herein, the nucleic acid encoding said TCR, the vector comprising said nucleic acid, the cell comprising said TCR for use in the treatment of cancer.

In one embodiment the cancer is a hematological cancer or a solid tumor.

Hematological cancers also called blood cancers which do not form solid tumors and therefore are dispersed in the body. Examples of hematological cancers are leukemia, lymphoma or multiple myeloma. There are two major types of solid tumors, sarcomas and carcinomas. Sarcomas are for example tumors of the blood vessel, bone, fat tissue, ligament, lymph vessel, muscle or tendon.

In one embodiment, the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and adenocarcinoma, cholangiocarcinoma, breast cancer, bladder cancer, myeloid leukemia and acute lymphoblastic leukemia, carcinoma, sarcoma or osteosarcoma.

Also contemplated herein are pharmaceutical compositions and kits containing one or more of (i) an isolated TCR as described herein; (ii) viral particles comprising a nucleic acid encoding a recombinant TCR; (iii) immune cells, such as T cells or NK cells, modified to express a recombinant TCR as described herein; (iv) nucleic acids encoding a recombinant TCR as described herein. In some embodiments, the present disclosure provides compositions comprising lentiviral vector particles comprising a nucleotide sequence encoding a recombinant TCR described herein (or T cells that have been modified using the vector particles described herein to express a recombinant TCR). Such compositions can be administered to subjects in the methods of the present disclosure as described further herein.

Compositions comprising the modified T cells as described herein can be utilized in methods and compositions for adoptive immunotherapy in accordance with known techniques, or variations thereof that will be apparent to those skilled in the art based on the instant disclosure.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

The number of cells for an effective treatment in the composition is typically greater than 10 cells, and up to 10⁶, up to and including 10⁸ or 10⁹ cells and can be more than 10¹⁰ cells. The number of cells will depend upon the ultimate use for which the composition is intended as will the type of cells included therein. For uses provided herein, the cells are generally in a volume of a liter or less, can be 500 ml or less, even 250 ml or 100 ml or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells. Pharmaceutical compositions provided herein can be in various forms, e.g., in solid, liquid, powder, aqueous, or lyophilized form. Examples of suitable pharmaceutical carriers are known in the art. Such carriers and/or additives can be formulated by conventional methods and can be administered to the subject at a suitable dose. Stabilizing agents such as lipids, nuclease inhibitors, polymers, and chelating agents can preserve the compositions from degradation within the body. In a composition intended to be administered by injection, one or more of a surfactant, preservative, wetting agent, dispersing agent, suspending agent, buffer, stabilizer and isotonic agent may be included.

The recombinant TCRs as described herein, or the viral vector particles comprising a nucleotide sequence encoding a recombinant TCR provided herein, can be packaged as kits. Kits can optionally include one or more components such as instructions for use, devices, and additional reagents, and components, such as tubes, containers and syringes for practice of the methods. Exemplary kits can include the nucleic acids encoding the recombinant TCRs, the recombinant TCR polypeptides, or viruses provided herein, and can optionally include instructions for use, a device for detecting a virus in a subject, a device for administering the compositions to a subject, and a device for administering the compositions to a subject.

Kits comprising polynucleotides encoding a gene of interest (e.g., a recombinant TCR) are also contemplated herein. Kits comprising a viral vector encoding a sequence of interest (e.g., a recombinant TCR) and optionally, a polynucleotide sequence encoding an immune checkpoint inhibitor are also contemplated herein.

Kits contemplated herein also include kits for carrying out the methods for detecting the presence of polynucleotides encoding any one or more of the TCRs disclosed herein. In particular, such diagnostic kits may include sets of appropriate amplification and detection primers and other associated reagents for performing deep sequencing to detect the polynucleotides encoding TCRs disclosed herein. In further embodiments, the kits herein may comprise reagents for detecting the TCRs disclosed herein, such as antibodies or other binding molecules. Diagnostic kits may also contain instructions for determining the presence of the polynucleotides encoding the TCRs disclosed herein or for determining the presence of the TCRs disclosed herein. A kit may also contain instructions. Instructions typically include a tangible expression describing the components included in the kit, and methods for administration, including methods for determining the proper state of the subject, the proper dosage amount, and the proper administration method. Instructions can also include guidance for monitoring the subject over the duration of the treatment time.

Kits provided herein also can include a device for administering a composition described herein to a subject. Any of a variety of devices known in the art for administering medications or vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a virus of the kit will be compatible with the virus of the kit; for example, a needle-less injection device such as a high pressure injection device can be included in kits with viruses not damaged by high pressure injection, but is typically not included in kits with viruses damaged by high pressure injection.

Kits provided herein also can include a device for administering a compound, such as a T cell activator or stimulator, or a TLR agonist, such as a TLR4 agonist to a subject. Any of a variety of devices known in the art for administering medications to a subject can be included in the kits provided herein. Exemplary devices include a hypodermic needle, an intravenous needle, a catheter, a needle-less injection, but are not limited to, a hypodermic needle, an intravenous needle, a catheter, a needle-less injection device, an inhaler, and a liquid dispenser such as an eyedropper. Typically, the device for administering the compound of the kit will be compatible with the desired method of administration of the compound.

### EXPERIMENTS

### Example 1: MAGE-A3-Reactive T Cell Clones Selectively Recognize the MAGE-A3_{EVD}-Peptide

An in vitro priming approach to isolate MAGE-A3-reactive T-cell clones was used. The priming system uses mature dendritic cells (mDCs) of HLA-A*01:01-positive as well as HLA-A*01:01-negative donors as antigen-presenting cells and autologous CD8⁺-enriched T cells as responding cells. In vitro transcribed RNA (ivtRNA) encoding the human MAGEA3 gene serves as the source of specific antigen. After electroporation into mDCs, the MAGE-A3-encoding ivtRNA is translated into protein, which is subsequently processed and presented as peptides by HLA-A*01:01-encoded molecules on the mDCs. For HLA-A*01:01-negative donors, ivtRNA coding for HLA-A*01:01 was used in addition to MAGE-A3 ivtRNA to transgenically express the respective HLA allele in the antigen-presenting cells (allogeneic approach). In vitro co-cultures of T cells with the ivtRNA-transfected mDCs from the same donor lead to de novo induction of antigen-specific T cells that serve as the source of corresponding TCRs. Antigen-specific T cells can be enriched by a variety of methods and are cloned by limiting dilution or FACS-based single cell sorting. Isolated and expanded MAGE-A3-reactive T cell clones were co-cultured with DU-145 cells previously transduced with HLA-A1-GFP (DU-145_A1) loaded with saturating amounts (10⁻⁵ M) of either MAGE-A3_{EVD}-peptide or the titin-derived control peptide (ESD). The titin-derived ESD-control peptide was cross-recognized by an affinity maturated MAGE-A3-reactive TCR leading to major cardiovascular toxicity during clinical testing (Brian J. Cameron, 2013; Linette et al., 2013). After 20-24 h, IFN-γ concentrations in co-culture supernatants were analyzed by standard sandwich ELISA (BD human IFN-γ ELISA set).

Results:

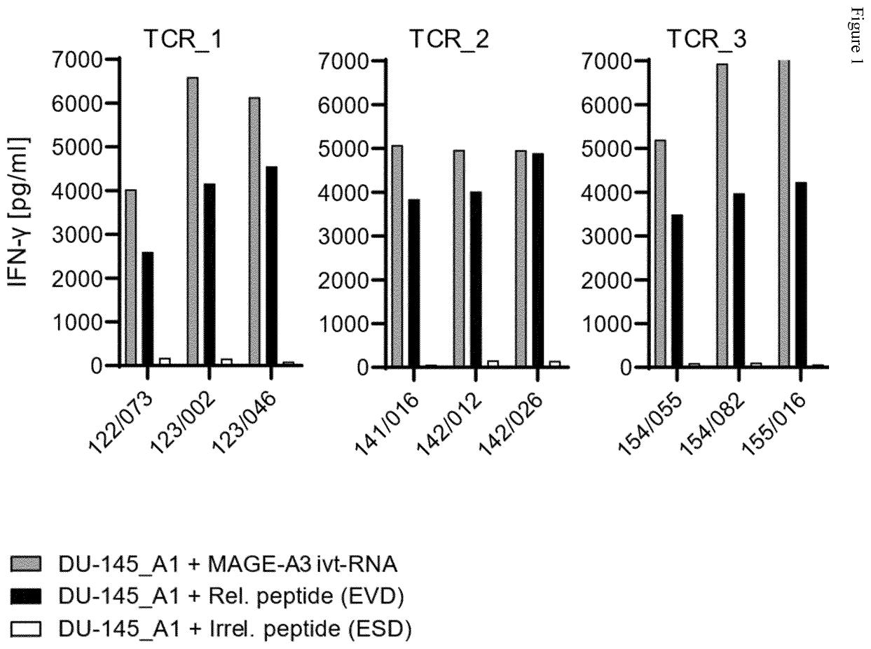

All MAGE-A3-reactive T cells clones selectively recognized the MAGE-A3_{EVD}-peptide while the titin_{ESD}-peptide was not recognized. Furthermore, tested T cell clones recognized endogenously processed and presented MAGE-A3_{EVD}-peptide in DU-145_A1 cells electroporated with ivtRNA encoding the human MAGEA3 gene. (FIG. 1).

### Example 2: Isolated MAGE-A3-Reactive TCRs can be Expressed Transgenically

Sequences of TCR-α and TCR-β chains of MAGE-A3-reactive T-cell clones were identified by Next Generation Sequencing and after exchanging the constant TCR regions by their minimal-murinized counterparts cloned into the retroviral vector pES.12-6. For the β chain the Cß1 sequence was used. PBMCs of three healthy donors were isolated by ficoll gradient centrifugation. CD8-positive Cß1-negative T-cells were enriched by negative magnetic selection (Miltenyi) and stimulated in non-tissue culture 24-well plates with plate bound antibodies specific for CD3 and CD28. Amphotropic retroviral particles were produced by transfection of HEK293T cells with the respective TCR encoding retroviral plasmid and two expression plasmids. Three days after stimulation, CD8-positive Cß1-negative T cells were transduced and on day ten enriched for transduced CD8⁺ cells by MACS-separation using the Cβ1 constant beta region as a marker for transduction and then expanded by REP. Transgenic T cells were stained either with an HLA-A1-restricted MAGE-A3_{EVD}-MHC-multimer (MAGE-A3, EVDPIGHLY; immuneAware) or antibodies against CD8 and the constant β1 region.

Results:

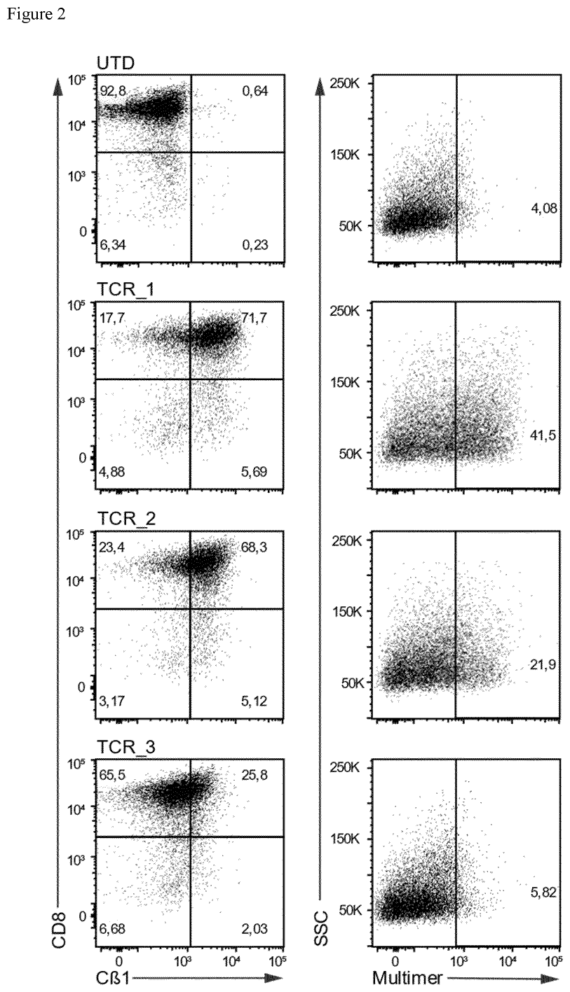

Transgenic T cell populations expressing TCR_1 and TCR_2 were efficiently transduced with around 70% Cß1-positive CD8-positive T cells. Both MAGE-A3-TCR-transgenic T cell populations bound the MAGE-A3_{EVD}-MHC-multimer efficiently (>40% for TCR_1 and 20% for TCR_2). Only 25% of T cell populations transduced with TCR_3 was positive for Cß1 indicating TCR expression and 5% of T cells bound the MAGE-A3_{EVD}-MHC-multimer. Very low MAGE-A3_{EVD}-MHC-multimer-staining and no Cß1 antibody staining was observed on untransduced CD8-positive T cells. These results show that TCRs isolated from MAGE-A3-reactive T-cell clones can be transgenically expressed in T cells of healthy donors. (FIG. 2).

### Example 3: T Cells Transgenically Expressing the TCRs Recognize Endogenously Processed and Presented Peptide

To assess target specificity of transgenic T cells DU-145 cells were previously transduced with HLA-A1-GFP and further engineered to express mCherry alone (negative control), MAGE-A3-mCherry or MAGE-A6-mCherry. MAGE-A3 and MAGE-A6 are highly homologous with 98% alignment of nucleotide and 95% alignment of protein sequences and therefore, cross-reactivity is likely (Newman et al., 2016). Untransduced (UTD) and transgenic T cells of three donors were co-cultured with transduced cells either unloaded or loaded with the relevant peptide (EVD). For recognition assays, the co-cultures are set-up at an effector-to-target ratio of 1:1, with 15,000 TCR-transgenic Cß1-positive T cells and 15,000 tumor cells. Recognition of target cells was analyzed by measuring the IFN-γ concentration in co-culture supernatants by a standard ELISA.

Results:

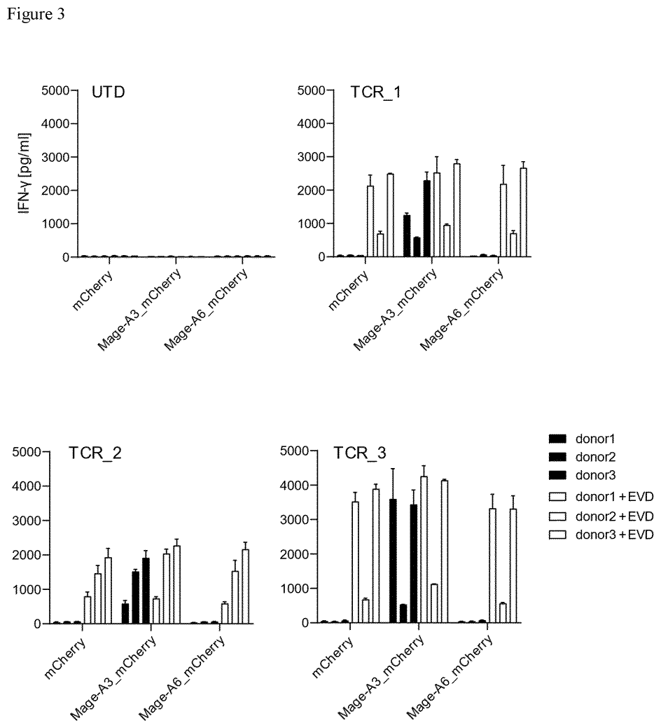

All TCR-transgenic T cell populations showed robust recognition of all peptide loaded target cells with donor dependent variations. Only MAGE-A3 engineered DU-145_A1 were recognized while MAGE-A6 overexpressing DU-145_A1 cells were not recognized. Untransduced T cells of all donors did not secrete IFN-γ in any of the tested conditions indicating no signs of activation. These results show that TCR_1, _2 and _3 selectively recognize MAGE-A3 but not a highly homologous family member like MAGE-A6. (FIG. 3).

### Example 4: MAGE-A3-TCR-Transgenic T Cells Recognize Endogenously MAGE-A3-Positive Tumor Cell Lines

Three tumor cell lines expressing titrated amounts of MAGE-A3 (SK-Mel23, MelA375, K562) and three MAGE-A3-negative tumor cell lines (OV-7, MS751, DU-145) are used for the experiment. Tumor cell lines were either endogenously HLA-A1-positive or engineered for HLA-A1 expression (K562, MS751, DU-145). All tumor cell lines were tested either unloaded or loaded with saturated concentrations of MAGE-A3_{EVD}-peptide (10⁻⁵ M). For recognition assays, the co-cultures are set-up at an effector-to-target ratio of about 1:1, with 15,000 TCR-transgenic Cß1-positive T cells and 15,000 tumor cells. Recognition of target cells was analyzed by measuring the IFN-γ concentration in co-culture supernatants by a standard ELISA.

Results:

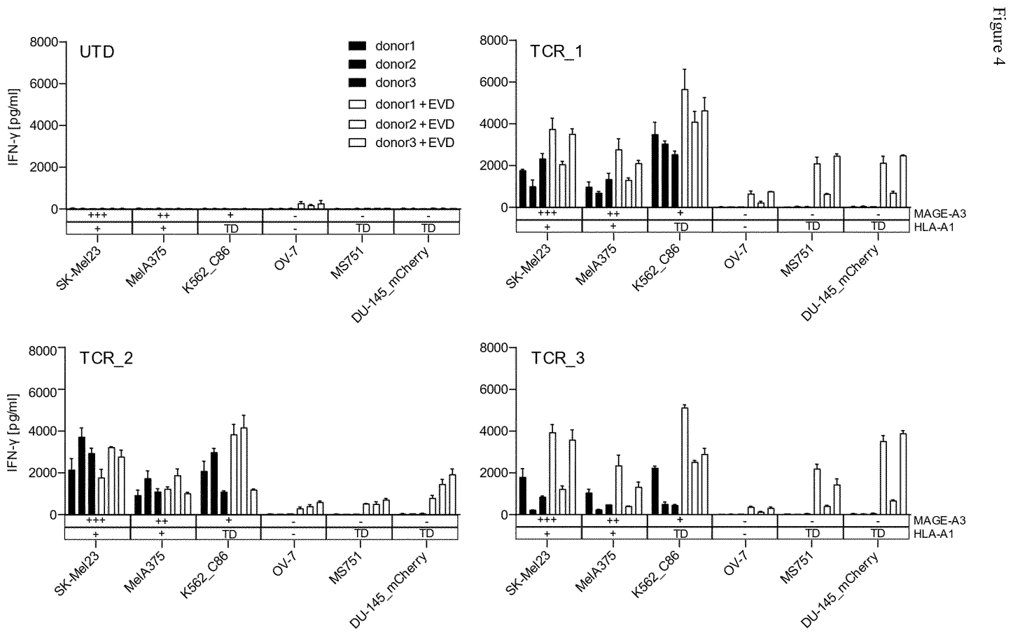

All TCR-transgenic T cell populations showed recognition of all peptide loaded target cells with donor dependent variations indicating sufficient HLA-A1 expression on target cells. TCR-transgenic T cell populations expressing TCR_1 and TCR_2 showed robust recognition of MAGE-A3 endogenous tumor cell lines with IFN-γ levels comparable to peptide loaded target cells representing the maximum possible IFN-γ release. TCR_3 transgenic T cells of donor 1 recognized MAGE-A3 expressing target cells to a comparable extent as TCR_1 and TCR_2 transgenic T cells while overall release of IFN-γ in donor 2 and donor 3 was lower. MAGE-A3-negative tumor cell lines were only recognized when peptide loaded. Untransduced T cells of all donors did not recognize any of the tested cell lines or conditions. These results show that MAGE-A3-TCR-transgenic T cells can efficiently recognize endogenously MAGE-A3-positive tumor cells in a highly selective manner. (FIG. 4).

The application further comprises the following items:

Item 1: Isolated T cell receptor (TCR) specific for MAGE-A3.

Item 2: Isolated TCR according to item 1, wherein the TCR specifically recognizes the amino acid sequence SEQ ID NO: 1 or a fragment thereof.

Item 3: Isolated TCR according to any one of the preceding embodiments, wherein the TCR does not recognize the amino acid sequence SEQ ID NO: 54 or a fragment thereof.

Item 4: Isolated TCR according to any of the preceding items, wherein the TCR specifically recognizes the amino acid sequence of SEQ ID NO: 1, which is presented by a molecule encoded by an HLA-A*01 gene.

Item 5: Isolated TCR according to any of the preceding items, wherein the TCR specifically recognizes the amino acid sequence of SEQ ID NO: 1, which is presented by an HLA-A*01:01 encoded molecule.

Item 6: Isolated TCR according to any one of the preceding items, wherein the TCR comprises
a)—a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
   a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; or
b)—a TCR α chain comprising a CDR1 having the amino acid sequence of SEQ ID NO: 10, a CDR2 having the amino acid sequence of SEQ ID NO: 11 and a CDR3 having the amino acid sequence of SEQ ID NO: 12, and
   a TCR β chain comprising a CDR1 having the amino acid sequence of SEQ ID NO: 13, a CDR2 having the amino acid sequence of SEQ ID NO: 14 and a CDR3 having the amino acid sequence of SEQ ID NO: 15; or
c)—a TCR α chain comprising a CDR1 having the amino acid sequence of SEQ ID NO: 18, a CDR2 having the amino acid sequence of SEQ ID NO: 19 and a CDR3 having the amino acid sequence of SEQ ID NO: 20, and
   a TCR β chain comprising a CDR1 having the amino acid sequence of SEQ ID NO: 21, a CDR 2 having the amino acid sequence of SEQ ID NO: 22 and a CDR 3 having the amino acid sequence of SEQ ID NO: 23.

Item 7: Isolated TCR according to any one of the preceding items, wherein the TCR comprises
a) a variable TCR α region having an amino acid sequence which is at least 80% identical to SEQ ID NO: 8 and a variable TCR β region having an amino acid sequence which is at least 80% identical to SEQ ID NO: 9; or
b) a variable TCR α region having an amino acid sequence which is at least 80% identical to SEQ ID NO: 16 and a variable TCR β region having an amino acid sequence which is at least 80% identical to SEQ ID NO: 17; or
c) a variable TCR α region having an amino acid sequence which is at least 80% identical to SEQ ID NO: 24 and a variable TCR β region having an amino acid sequence which is at least 80% identical to SEQ ID NO: 25.

Item 8: Isolated TCR according to any one of the preceding embodiments, wherein the TCR comprises
a) a variable TCR α region having the amino acid sequence of SEQ ID NO: 8 and a variable TCR β region having the amino acid sequence of SEQ ID NO: 9; or
b) a variable TCR α region having the amino acid sequence of SEQ ID NO: 16 and a variable TCR β region having the amino acid sequence of SEQ ID NO: 17; or
c) a variable TCR α region having the amino acid sequence of SEQ ID NO: 24 and a variable TCR β region having the amino acid sequence of SEQ ID NO: 25.

Item 9: Isolated TCR according to any one of the preceding items, wherein the TCR comprises a constant TCR α region having an amino acid sequence which is at least 80% identical to SEQ ID NO: 50 and a constant TCR β region having an amino acid sequence which is at least 80% identical to SEQ ID NO: 51.

Item 10: Isolated TCR according to any one of the preceding items, wherein the TCR comprises a constant TCR α region having the amino acid sequence of SEQ ID NO: 50 and a constant TCR β region having the amino acid sequence of SEQ ID NO: 51.

Item 11: Isolated TCR according to any one of the preceding items, wherein the TCR is purified.

Item 12: Isolated TCR according to any one of the preceding items, wherein its amino acid sequence comprises one or more phenotypically silent substitutions.

Item 13: Isolated TCR according to any one of the preceding items, wherein its amino acid sequence is modified to comprise a detectable label, a therapeutic agent or pharmacokinetic modifying moiety.

Item 14: Isolated TCR according to item 13, wherein the therapeutic agent is selected from the group consisting of an immune effector molecule, a cytotoxic agent and a radionuclide.

Item 15: Isolated TCR according to item 14, wherein the immune effector molecule is a cytokine.

Item 16: Isolated TCR according to any one of the preceding items, wherein the TCR is soluble or membrane bound.

Item 17: Isolated TCR according to item 13, wherein the pharmacokinetic modifying moiety is at least one polyethylene glycol repeating unit, at least one glycol group, at least one sialyl group or a combination thereof.

Item 18: Isolated TCR according to any one of the preceding items, wherein the TCR is of the single chain type, wherein the TCR α chain and the TCR β chain are linked by a linker sequence.

Item 19: Isolated TCR according to any one of the preceding items, wherein the TCR α chain or the TCR β chain is modified to comprise an epitope tag.

Item 20: Isolated polypeptide comprising a functional portion of the TCR of any one of the preceding items, wherein the functional portion comprises the
d) the amino acid sequences SEQ ID NOs: 2, 3, 4, 5, 6, and 7, or
e) the amino acid sequences SEQ ID NOs: 10, 11, 12, 13, 14, and 15, or
f) the amino acid sequences SEQ ID NOs: 18, 19, 20, 21, 22 and 23.

Item 21: Isolated polypeptide according to item 20, wherein the functional portion comprises the
a) a variable TCR α region having the amino acid sequence of SEQ ID NO: 8 and a variable TCR β region having the amino acid sequence of SEQ ID NO: 9; or
b) a variable TCR α region having the amino acid sequence of SEQ ID NO: 16 and a variable TCR β region having the amino acid sequence of SEQ ID NO: 17; or
c) a variable TCR α region having the amino acid sequence of SEQ ID NO: 24 and a variable TCR β region having the amino acid sequence of SEQ ID NO: 25.

Item 22: Multivalent TCR complex comprising at least one of the TCRs as embodied in any one of items 1 to 19.

Item 23: Multivalent TCR complex comprising at least two of the TCRs as embodied in any one of items 1 to 19.

Item 24: Isolated TCR according to items 1 to 19, polypeptide according to items 20 to 21, multivalent TCR complex according to items 22 to 23, wherein IFN-γ secretion is induced by binding to the amino acid sequence of SEQ ID NO: 1, which is presented by the HLA-A*01:01 encoded molecule.

Item 25: Nucleic acid encoding a TCR according to any one of items 1 to 19 or encoding the polypeptide according to items 20 to 21.

Item 26: Vector comprising the nucleic acid of item 25.

Item 27: Vector according to item 26, wherein the vector is an expression vector.

Item 28: Vector according to item 26 or 27, wherein the vector is a retroviral vector.

Item 29: Vector according to item 26 or 27, wherein the vector is a lentiviral vector.

Item 30: Cell expressing the TCR according to items 1 to 19 or the isolated polypeptide according to items 20 or 21.

Item 31: Cell according to item 30, wherein the cell is isolated or non-naturally occurring.

Item 32: Cell according to items 30 or 331, wherein the cell comprises the nucleic acid according to item 25 or the vector according to items 26 to 29.

Item 33: Cell according to items 30 to 32, wherein the cell comprises:
a) an expression vector which comprises at least one nucleic acid as embodied in item 25, or
b) a first expression vector which comprises a nucleic acid encoding the alpha chain of the TCR as embodied in any one of the items 1 to 19, and a second expression vector which comprises a nucleic acid encoding the beta chain of a TCR as embodied in any one of the items 1 to 19.

Item 34: Cell according to any one of items 30 to 33, wherein the cell is a peripheral blood lymphocyte (PBL) or a peripheral blood mononuclear cell (PBMC).
Item 35: Cell according to any one of items 30 to 34, wherein the cell is a T cell.

Item 36: Antibody or antigen binding fragment thereof specifically binding to a portion of the TCR according to items 1 to 19 that mediates specificity for MAGE-A3.

Item 37: Antibody according to item 36, wherein the portion of the TCR that mediates the MAGE-A3 specificity comprises
d) CDR3 of the alpha chain of SEQ ID NO: 4 and/or the CDR3 of the beta chain of SEQ ID NO: 7 or;
e)) CDR3 of the alpha chain of SEQ ID NO: 12 and/or the CDR3 of the beta chain of SEQ ID NO: 15 or;
f) CDR3 of the alpha chain of SEQ ID NO: 20 and/or the CDR3 of the beta chain of SEQ ID NO: 23.

Item 38: Pharmaceutical composition comprising the TCR according to items 1 to 19, the polypeptide according to items 20 to 22, the multivalent TCR complex according to item 22 to 23 the nucleic acid according to item 25, the vector according to items 26 to 29, the cell according to any one of items 30 to 35, or the antibody according to items 36 to 37.

Item 39: Pharmaceutical composition according to item 38 wherein the pharmaceutical composition comprises at least one pharmaceutically acceptable carrier.

Item 40: The TCR according to items 1 to 19, the polypeptide according to items 20 to 21, the multivalent TCR complex according to item 22 to 23, the nucleic acid according to item 25, the vector according to items 26 to 29, the cell according to any one of items 30 to 31, or the antibody according to items 36 to 37 for use as a medicament.

Item 41: The TCR according to items 1 to 19, the polypeptide according to items 20 to 21, the multivalent TCR complex according item 22 to 23, the nucleic acid according to item 25, the vector according to claims 26 to 27 or the cell according to any one of items 30 to 35 for use in the treatment of cancer.

Item 42: The TCR, the polypeptide, the multivalent TCR complex, the nucleic acid, the vector or the cell for use according to item 41, wherein the cancer is a hematological cancer or a solid tumor.

Item 43: The TCR, the polypeptide, the multivalent TCR complex, the nucleic acid, the vector or the cell for use according to items 41 and 42, wherein the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and neck cancer, gastric cancer, endometrial cancer, cervical cancer, colorectal cancer, stomach adenocarcinoma, cholangiocarcinoma, breast cancer, bladder cancer, myeloid leukemia and acute lymphoblastic leukemia, carcinoma, sarcoma or osteosarcoma.

Item 44: The TCR, the polypeptide, the multivalent TCR complex, the nucleic acid, the vector or the cell for use according to item 43, wherein the cancer is preferably selected from the group consisting sarcoma or carcinoma.

1. Isolated T cell receptor (TCR) specific for MAGE-A3.

2. Isolated TCR according to claim 1, wherein the TCR specifically recognizes the amino acid sequence SEQ ID NO: 1 or a fragment thereof.

3. Isolated TCR according to claim 1, wherein the TCR does not recognize the amino acid sequence SEQ ID NO: 54 or a fragment thereof.

4. Isolated TCR according to any one of the preceding claims, wherein the TCR specifically recognizes the HLA-A1 bound form of the amino acid sequence of SEQ ID NO: 1, preferably wherein the TCR specifically recognizes the amino acid sequence of SEQ ID NO: 1, which is presented by the HLA-A*01:01-encoded molecule.

5. Isolated TCR according to any one of the preceding claims, wherein the TCR comprises
a)—a TCR α chain comprising a CDR1 having the amino acid sequence of SEQ ID NO: 2, a CDR2 having the amino acid sequence of SEQ ID NO: 3 and a CDR3 having the amino acid sequence of SEQ ID NO: 4, and
a TCR β chain comprising a CDR1 having the amino acid sequence of SEQ ID NO: 5, a CDR2 having the amino acid sequence of SEQ ID NO: 6 and a CDR3 having the amino acid sequence of SEQ ID NO: 7; or
b)—a TCR α chain comprising a CDR1 having the amino acid sequence of SEQ ID NO: 10, a CDR2 having the amino acid sequence of SEQ ID NO: 11 and a CDR3 having the amino acid sequence of SEQ ID NO: 12, and
a TCR β chain comprising a CDR1 having the amino acid sequence of SEQ ID NO: 13, a CDR2 having the amino acid sequence of SEQ ID NO: 14 and a CDR3 having the amino acid sequence of SEQ ID NO: 15; or
c)—a TCR α chain comprising a CDR1 having the amino acid sequence of SEQ ID NO: 18, a CDR2 having the amino acid sequence of SEQ ID NO: 19 and a CDR3 having the amino acid sequence of SEQ ID NO: 20, and
a TCR β chain comprising a CDR1 having the amino acid sequence of SEQ ID NO: 21, a CDR 2 having the amino acid sequence of SEQ ID NO: 22 and a CDR 3 having the amino acid sequence of SEQ ID NO: 23.

6. Isolated TCR according to any one of the preceding claims, wherein the TCR
a) a variable TCR α region having an amino acid sequence which is at least 80% identical to SEQ ID NO: 8 and a variable TCR β region having an amino acid sequence which is at least 80% identical to SEQ ID NO: 9; or
b) a variable TCR α region having an amino acid sequence which is at least 80% identical to SEQ ID NO: 16 and a variable TCR β region having an amino acid sequence which is at least 80% identical to SEQ ID NO: 17; or
c) a variable TCR α region having an amino acid sequence which is at least 80% identical to SEQ ID NO: 24 and a variable TCR β region having an amino acid sequence which is at least 80% identical to SEQ ID NO: 25.

7. Isolated polypeptide comprising a functional portion of the TCR of any of claims 1 to 6, wherein the functional portion comprises the
a) the amino acid sequences SEQ ID NOs: 2, 3, 4, 5, 6, and 7, or
b) the amino acid sequences SEQ ID NOs: 10, 11, 12, 13, 14, and 15, or
c) the amino acid sequences SEQ ID NOs: 18, 19, 20, 21, 22 and 23.

8. Multivalent TCR complex comprising a least one TCR as claimed in any one of claims 1 to 6.

9. Isolated TCR according to claims 1 to 6, isolated polypeptide according to claim 7, multivalent TCR complex according to claim 8, wherein IFN-γ secretion is induced by binding to the amino acid sequence of SEQ ID NO: 1, which is presented by the HLA-A*01:01-encoded molecule.

10. Nucleic acid encoding a TCR according to any one of claims 1 to 6 or encoding the isolated polypeptide according to claim 7.

11. Vector comprising the nucleic acid of claim 10, wherein the vector is preferably an expression vector, more preferably a retroviral vector or lentiviral vector.

12. Cell expressing the TCR according to claims 1 to 6.

13. Antibody or antigen binding fragment thereof specifically binding to a portion of the TCR according to claims 1 to 6, wherein the portion of the TCR comprises
a) the CDR3 of the alpha chain of SEQ ID NO: 4 and/or the CDR3 of the beta chain of SEQ ID NO: 7 or;
b) the CDR3 of the alpha chain of SEQ ID NO: 12 and/or the CDR3 of the beta chain of SEQ ID NO: 15 or;
c) the CDR3 of the alpha chain of SEQ ID NO: 20 and/or the CDR3 of the beta chain of SEQ ID NO: 23.

14. The TCR according to claims 1 to 6, the polypeptide according to claim 7, the multivalent TCR complex according to claim 8, the nucleic acid according to claim 10, the vector according to claim 11, the cell according to claim 12, or the antibody according to claim 13 for use as a medicament.

15. The TCR according to claims 1 to 6, the polypeptide according to claim 7, the multivalent TCR complex according to claim 8, the nucleic acid according to claim 10, the vector according to claim 11, the cell according to claim 12, or the antibody according to claim 13 for use in the treatment of cancer, wherein the cancer is selected from the group consisting of prostate cancer, uterine cancer, thyroid cancer, testicular cancer, renal cancer, pancreatic cancer, ovarian cancer, esophageal cancer, non-small-cell lung cancer, lung adenocarcinoma, squamous cell carcinoma, non-Hodgkin's lymphoma, multiple myeloma, melanoma, hepatocellular carcinoma, head and adenocarcinoma, cholangiocarcinoma, breast cancer, bladder cancer, myeloid leukemia and acute lymphoblastic leukemia, carcinoma, sarcoma or osteosarcoma.


SEQUENCE LISTING (54 sequences)

SEQ ID NO: 1 - protein, 9 aa, Homo sapiens
EVDPIGHLY

SEQ ID NO: 2 - protein, 6 aa, Homo sapiens
DSAIYN

SEQ ID NO: 3 - protein, 7 aa, Homo sapiens
IQSSQRE

SEQ ID NO: 4 - protein, 12 aa, Homo sapiens
CAVIEYGNKLVF

SEQ ID NO: 5 - protein, 5 aa, Homo sapiens
LNHNV

SEQ ID NO: 6 - protein, 6 aa, Homo sapiens
YYDKDF

SEQ ID NO: 7 - protein, 13 aa, Homo sapiens
CATSWDRGYEQFF

SEQ ID NO: 8 - protein, 131 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVIEYGNKLVFGAGTILRVKSY

SEQ ID NO: 9 - protein, 131 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSWDRGYEQFFGPGTRLTVL

SEQ ID NO: 10 - protein, 6 aa, Homo sapiens
DSAIYN

SEQ ID NO: 11 - protein, 7 aa, Homo sapiens
IQSSQRE

SEQ ID NO: 12 - protein, 14 aa, Homo sapiens
CAVRWETSGSRLTF

SEQ ID NO: 13 - protein, 5 aa, Homo sapiens
SGDLS

SEQ ID NO: 14 - protein, 6 aa, Homo sapiens
YYNGEE

SEQ ID NO: 15 - protein, 13 aa, Homo sapiens
CASSVGGGYEQYF

SEQ ID NO: 16 - protein, 133 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRWETSGSRLTFGEGTQLTVNPD

SEQ ID NO: 17 - protein, 131 aa, Homo sapiens
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSVGGGYEQYFGPGTRLTVT

SEQ ID NO: 18 - protein, 7 aa, Homo sapiens
TRDTTYY

SEQ ID NO: 19 - protein, 8 aa, Homo sapiens
RNSFDEQN

SEQ ID NO: 20 - protein, 17 aa, Homo sapiens
CALSEALGYSSASKIIF

SEQ ID NO: 21 - protein, 5 aa, Homo sapiens
SGHTA

SEQ ID NO: 22 - protein, 6 aa, Homo sapiens
FQGNSA

SEQ ID NO: 23 - protein, 16 aa, Homo sapiens
CASSDPMSGAQETQYF

SEQ ID NO: 24 - protein, 139 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEALGYSSASKIIFGSGTRLSIRPN

SEQ ID NO: 25 - protein, 135 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSDPMSGAQETQYFGPGTRLLVL

SEQ ID NO: 26 - DNA, 18 bp, Artificial Sequence
  codon optimized
gacagcgccatctacaac

SEQ ID NO: 27 - DNA, 21 bp, Artificial Sequence
  codon optimized
attcagagcagccagagagag

SEQ ID NO: 28 - DNA, 36 bp, Artificial Sequence
  codon optimized
tgcgccgtgatcgagtacggcaacaagctggtgttt

SEQ ID NO: 29 - DNA, 15 bp, Artificial Sequence
  codon optimized
ctgaaccacaacgtg

SEQ ID NO: 30 - DNA, 18 bp, Artificial Sequence
  codon optimized
tactacgacaaggacttc

SEQ ID NO: 31 - DNA, 39 bp, Artificial Sequence
  codon optimized
tgtgccaccagctgggatagaggctacgagcagtttttc

SEQ ID NO: 32 - DNA, 393 bp, Artificial Sequence
  codon optimized
atggagacactgctgggactgctgattctgtggctgcagcttcagtgggtgtccagcaagcaagaagtgacacagatccctgccgctctgtctgtgcctgagggcgaaaacctggtgctgaactgctccttcaccgacagcgccatctacaacctgcagtggttcagacaggaccccggcaagggactgacaagcctgctgctcattcagagcagccagagagagcagaccagcggcagactgaatgccagcctggataagtcctccggcagaagcaccctgtatatcgccgcttctcagcctggcgatagcgccacatatctgtgcgccgtgatcgagtacggcaacaagctggtgtttggcgccggaaccatcctgagagtgaagtcctat

SEQ ID NO: 33 - DNA, 393 bp, Artificial Sequence
  codon optimized
atgggacctggactgctgcattggatggccctgtgtctgctcggaacaggacatggcgacgctatggtcattcagaaccccagataccaagtgacccagttcggcaagcccgtgacactgagctgtagccagacactgaaccacaacgtgatgtactggtatcagcagaagtcctctcaggcccctaagctgctgttccactactacgacaaggacttcaacaacgaggccgacacacccgacaacttccagagcagaaggcccaataccagcttctgcttcctggacatcagaagccctggcctgggagatgccgccatgtatctgtgtgccaccagctgggatagaggctacgagcagtttttcggccctggcaccagactgacagtgctg

SEQ ID NO: 34 - DNA, 18 bp, Artificial Sequence
  codon optimized
gacagcgccatctacaac

SEQ ID NO: 35 - DNA, 21 bp, Artificial Sequence
  codon optimized
attcagagcagccagagagag

SEQ ID NO: 36 - DNA, 42 bp, Artificial Sequence
  codon optimized
tgcgccgtcagatgggagacaagcggctccagactgacattt

SEQ ID NO: 37 - DNA, 15 bp, Artificial Sequence
  codon optimized
agcggcgacctgagc

SEQ ID NO: 38 - DNA, 18 bp, Artificial Sequence
  codon optimized
tactacaacggcgaggaa

SEQ ID NO: 39 - DNA, 39 bp, Artificial Sequence
  codon optimized
tgtgcctcttctgtcggcggaggctacgagcagtatttc

SEQ ID NO: 40 - DNA, 399 bp, Artificial Sequence
  codon optimized
atggagacactgctgggactgctgattctgtggctgcagcttcagtgggtgtccagcaagcaagaagtgacacagatccctgccgctctgtctgtgcctgagggcgaaaacctggtgctgaactgctccttcaccgacagcgccatctacaacctgcagtggttcagacaggaccccggcaagggactgacaagcctgctgctcattcagagcagccagagagagcagaccagcggcagactgaatgccagcctggataagtcctccggcagaagcaccctgtatatcgccgcttctcagcctggcgatagcgccacatatctgtgcgccgtcagatgggagacaagcggctccagactgacatttggcgagggcacacagctgaccgtgaatcccgat

SEQ ID NO: 41 - DNA, 393 bp, Artificial Sequence
  codon optimized
atgggcttcagactgctgtgctgcgtggccttttgtctgcttggagccggacctgtggatagcggcgttacccagacacctaagcacctgatcacagccacaggccagcgcgtgaccctgagatgttctcctagaagcggcgacctgagcgtgtactggtatcagcagtctctggaccagggcctgcagttcctgatccagtactacaacggcgaggaaagagccaagggcaacatcctggaacggttcagcgcccagcagttcccagatctgcacagcgagctgaacctgagcagcctggaactgggagatagcgccctgtacttctgtgcctcttctgtcggcggaggctacgagcagtatttcggccctggcaccagactgaccgtgaca

SEQ ID NO: 42 - DNA, 21 bp, Artificial Sequence
  codon optimized
acacgggacaccacctactac

SEQ ID NO: 43 - DNA, 24 bp, Artificial Sequence
  codon optimized
cggaacagcttcgacgagcagaac

SEQ ID NO: 44 - DNA, 51 bp, Artificial Sequence
  codon optimized
tgcgccctgtctgaggccctgggctacagctctgccagcaagatcatcttt

SEQ ID NO: 45 - DNA, 15 bp, Artificial Sequence
  codon optimized
agcggacacacagcc

SEQ ID NO: 46 - DNA, 18 bp, Artificial Sequence
  codon optimized
ttccaaggcaacagcgcc

SEQ ID NO: 47 - DNA, 48 bp, Artificial Sequence
  codon optimized
tgtgccagcagcgatcctatgtctggcgcccaagagacacagtacttc

SEQ ID NO: 48 - DNA, 417 bp, Artificial Sequence
  codon optimized
atgctgacagcctctctgctgagagccgtgatcgccagcatctgtgtggtgtctagcatggcccagaaagtgacacaggcccagaccgagatcagcgtggtggaaaaagaagatgtgaccctggactgcgtgtacgagacacgggacaccacctactacctgttctggtacaagcagcctcctagcggcgagctggtgttcctgatcagacggaacagcttcgacgagcagaacgagatctccggccggtacagctggaacttccagaagtccaccagcagcttcaacttcaccatcaccgccagccaggtggtggatagcgccgtgtatttttgcgccctgtctgaggccctgggctacagctctgccagcaagatcatctttggcagcggcacccggctgagcatcagacctaat

SEQ ID NO: 49 - DNA, 405 bp, Artificial Sequence
  codon optimized
atgggcaccagactgctgttctgggtcgccttttgtctgctgggcgccgatcatacaggtgccggtgtttctcagagccccagcaacaaagtgaccgagaagggcaaagacgtggaactgagatgcgaccccatcagcggacacacagccctgtactggtacagacagtctctcggccagggcctcgagttcctgatctacttccaaggcaacagcgcccctgacaagagcggcctgcctagcgatagattcagcgccgaaagaacaggcggcagcgtgtccacactgaccatccagagaacccagcaagaggacagcgccgtgtacctgtgtgccagcagcgatcctatgtctggcgcccaagagacacagtacttcggccctggaacacggctgctggttctt

SEQ ID NO: 50 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 51 - protein, 177 aa, Homo sapiens
EDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 52 - DNA, 420 bp, Artificial Sequence
  codon optimized
atccagaatccggaccccgccgtgtaccagctgcgcgacagcaagagcagcgacaagagcgtgtgcctgttcaccgacttcgacagccagaccaacgtgagccagagcaaggacagcgacgtgtacatcaccgacaagaccgtgctggacatgcgcagcatggacttcaagagcaacagcgccgtggcctggagcaacaagagcgacttcgcctgcgccaacgccttcaacaacagcatcatccccgaggacaccttcttccccagcagcgatgtgccgtgcgacgtgaagctggtggagaagagcttcgagaccgacaccaacctgaacttccagaacctgagcgtgatcggcttccgcatcctgctgctgaaggtggccggcttcaacctgctgatgaccctgcgcctgtggagcagc

SEQ ID NO: 53 - DNA, 531 bp, Artificial Sequence
  codon optimized
gaggacctgaacaaggtgttccctccggaggtggccgtgttcgagcccagcaaagccgagatcgcgcacacccagaaggccaccctggtgtgcctggccaccggcttcttccccgaccacgtggagctgagctggtgggtgaacggcaaggaggtgcacagcggcgtgagcaccgacccccagcccctgaaggagcagcccgccctgaacgacagccgctactgcctgagcagccgcctgcgcgtgagcgccaccttctggcagaacccccgcaaccacttccgctgccaggtgcagttctacggcctgagcgagaacgacgagtggacccaggaccgcgccaagcccgtgacccagatcgtgagcgccgaggcctggggccgcgccgactgcggcattaccagcgcgagctaccatcagggcgtgctgagcgccaccatcctgtacgagatcctgctgggcaaggccaccctgtacgccgtgctggtgagcgccctggtgctgatggcgatggtgaagcgcaaggacttc

SEQ ID NO: 54 - protein, 9 aa, Homo sapiens
ESDPIVAQY